(19) United States
(12) Patent Application Publication
(10) Pub. No.: US 2021/0322354 A1
(43) Pub. Date: Oct. 21, 2021

(54) METHODS OF TREATING INFLAMMATORY, FIBROTIC, AND PROLIFERATIVE CONDITIONS USING COMPOSITIONS COMPRISING DGLA AND/OR 15-HETRE, AND ASSOCIATED METHODS AND COMPOSITIONS

(71) Applicant: DS Biopharma Limited, Dublin (IE)

(72) Inventors: John Climax, Dublin (IE); David Coughlan, Dublin (IE); Markus Weissbach, Dublin (IE); Moayed Hamza, Dublin (IE)

(21) Appl. No.: 17/106,348

(22) Filed: Nov. 30, 2020

Related U.S. Application Data
(63) Continuation of application No. 16/886,308, filed on May 28, 2020, now abandoned.
Application No. 16/886,308 is a continuation of application No. 16/653,004, filed on Oct. 15, 2019, now abandoned.
(60) Provisional application No. 62/747,539, filed on Oct. 18, 2018.

(51) Int. Cl. A61K 31/202; A61P 17/00; A61P 37/06

(52) U.S. Cl. CPC A61K 31/202; A61P 37/06; A61P 17/00

(57) ABSTRACT

The present disclosure provides orally and/or topically deliverable pharmaceutical compositions comprising DGLA or a derivative thereof and/or 15-HETrE or a derivative thereof and methods of using same to treat a variety of conditions, diseases, and disorders, including but not limited to inflammatory, fibrotic, and proliferative conditions, diseases, and disorders.

### PRIORITY CLAIM

This application is a continuation of U.S. patent application Ser. No. 16/886,308, filed on May 28, 2020, which is a continuation of U.S. patent application Ser. No. 16/953,004, filed on Oct. 15, 2019, which claims priority to U.S. Provisional Patent Application No. 62/747,539, filed on Oct. 18, 2018, the entire contents of which is incorporated herein by reference.

### TECHNICAL FIELD

The present application relates generally to methods of treating inflammatory, fibrotic, and proliferative conditions using compositions comprising DGLA and/or 15-HETrE, and methods and compositions of using the same.

### BACKGROUND

Dihomo gamma linolenic acid (DGLA) is an essential fatty acid found naturally in the body as the elongation product of gamma linolenic acid (GLA). GLA is, in turn, a desaturation product of linoleic acid. Soft gelatin encapsulation of DGLA is challenging as it is prone to oxidation to aldehydes, which can interact with amino groups in the gelatin polymer in the capsule shell. This can cause slowdown in drug release due to crosslinking of the gelatin polymers.

15(S)-hydroxyeicosatrienoic acid (15S-HETrE; 15-hydroxy-8(Z), 11(Z), 13(E)-eicosatrienoic acid) is a metabolite of gamma-linolenic acid, a polyunsaturated fatty acid reported to modulate arachidonic acid (AA) metabolism. 15S-HETrE suppresses expression of cyclooxygenase-2 (COX-2) and synthesis of prostaglandin E2 (PGE2).

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic diagram of the study described in Example 2 and its duration.

FIGS. 2A-2C are sections of a heat map showing an effect of DS107 (drug) at two doses (1 g or 2 g) or a placebo on expression of biomarkers in subjects following administration of Drug1g, Drug2g, or Placebo.

FIG. 3 shows box plots illustrating fold change in expression of general inflammatory markers, such as TRAIL, IL1RT2, IL1RT1/1L1R1, MMP-1, MMP-10, MMP-7, and TNFRSF9 following administration of Drug1g, Drug2g, or Placebo to a subject relative to baseline.

FIG. 4 shows box plots illustrating fold change in expression of innate immunity related markers, such as CD163, IL6RA, GRN, MARCO, PGLyRP1, and CSF.1 following administration of Drug1g, Drug2g, or Placebo to a subject relative to baseline.

FIGS. 5A and 5B show box plots illustrating fold change in expression of T cell and natural killer (NK) cell activation markers, such as IL-15RA, IL-1, IL-2RB, CCL23, IL-7, CD6, SLAMF1, CD244, CD40, and ALCAM following administration of Drug1g, Drug2g, or Placebo to a subject relative to baseline.

FIGS. 6A and 6B show box plots illustrating fold change in expression of Th1-related markers, such as CCL16, CCL3, CXCL9, IL-18, IL-18R1, IL-18BP, CCL-3/MIP-1-alpha, and CCL-4 following administration of Drug1g, Drug2g, or Placebo to a subject relative to baseline.

FIGS. 7A and 7B show box plots illustrating fold change in expression of Th17/Th1-related markers, such as PI3/Elafin, CDCP1/CD318, IL-17A, IL-17D, IL-12B, IL-17RA, IL-20, CXCL1, and IL-12 following administration of Drug1g, Drug2g, or Placebo to a subject relative to baseline.

FIG. 8 shows box plots illustrating fold change in expression of Th2-related markers, such as IL-5Ralpha, IL-10, IL-10RB, and IL-13 following administration of Drug1g, Drug2g, or Placebo to a subject relative to baseline.

FIG. 9 shows box plots illustrating fold change in expression of Th2-related markers, chemokines, and adhesion molecules, such as CCL-23, CCL-24, CCL-28, IL-24, CCL-11, and VEGF-A following administration of Drug1g, Drug2g, or Placebo to a subject relative to baseline

FIGS. 10A and 10B show box plots illustrating fold change in expression of fibrotic and proliferative markers, such as TGF-alpha, PDGF-A, GDF-15, EGFR, VEGF-A, HGF, AXL, and TGF-B following administration of Drug1g, Drug2g, or Placebo to a subject relative to baseline.

### SUMMARY

The present disclosure provides orally and/or topically deliverable pharmaceutical compositions comprising DGLA or a derivative thereof and/or 15-HETrE or a derivative thereof, and methods of using same to treat a variety of conditions and disorders.

In one aspect, the present disclosure provides methods of treating liver fibrosis and/or a non-fatty liver disease or disorder in a subject in need thereof, the method comprising administering to the subject a pharmaceutical composition comprising DGLA or a derivative thereof, 15-HETrE or a derivative thereof, or a combination thereof.

In one embodiment, the non-fatty liver disease or disorder is selected from the group consisting of alcoholic hepatitis, primary sclerosing cholangitis, primary biliary cholangitis, viral hepatitis, autoimmune hepatitis, iatrogenic-induced livery injury, and hepatic veno-occlusive disease. In another embodiment, the viral hepatitis is caused by hepatitis B virus and/or hepatitis C virus. In still yet another embodiment, the viral hepatitis is chronic. In a further embodiment, the iatrogenic-induced liver injury is a drug-induced liver injury. In still a further embodiment, the method further comprises reducing an amount of one or more cytokines and/or chemokines in the subject in need thereof. In yet another embodiment, the one or more cytokines and/or chemokines are selected from the group consisting of transforming growth factor α (TGF-α), transforming growth factor β (TGF-β), epidermal growth factor receptor (EGFR), vascular endothelial growth factor subunit A (VEGF-A), and tyrosine-protein kinase receptor UFO (AXL). In still another embodiment, the one or more cytokines and/or chemokines are TGF-β and AXL. In yet another embodiment, the non-fatty liver disease is alcoholic hepatitis and/or viral hepatitis. In an additional embodiment, the one or more cytokines and/or chemokines are TGF-β and EGFR. In yet an additional embodiment, the non-fatty liver disease is primary sclerosing cholangitis and/or primary biliary cholangitis. In still an additional embodiment, the one or more cytokines and/or chemokines is TGF-β. In a further embodiment, the non-fatty liver disease is autoimmune hepatitis and/or iatrogenic-induced liver injury. In yet another additional embodiment, the one or more cytokines and/or chemokines are TGF-β and VEGF-A. In still yet another additional embodiment, the non-fatty liver disease is hepatic veno-occlusive disease.

In another aspect, the present disclosure provides methods of treating an ocular disease or disorder in a subject in need thereof, the method comprising administering to the subject a pharmaceutical composition comprising DGLA or a derivative thereof, 15-HETrE or a derivative thereof, or a combination thereof.

In another embodiment, the ocular disease or disorder is selected from the group consisting of corneal opacification, glaucoma, age-related macular degeneration, and cataract. In still a further embodiment, the method further comprises reducing an amount of one or more cytokines and/or chemokines in the subject in need thereof. In yet another embodiment, the one or more cytokines and/or chemokines is TGF-β and/or VEGF-A.

In yet another aspect, the present disclosure provides methods of treating a connective tissue disease or disorder in a subject in need thereof, the method comprising administering to the subject a pharmaceutical composition comprising DGLA or a derivative thereof, 15-HETrE or a derivative thereof, or a combination thereof.

In yet another embodiment, the method further comprises reducing an amount of TGF-β in the subject in need thereof. In yet another embodiment, the connective tissue disease or disorder is selected from the group consisting of Marfan's syndrome, Loeys-Dietz syndrome, and vascular Ehlers-Danlos syndrome.

In a further aspect, the present disclosure provides methods of treating Alzheimer's disease in a subject in need thereof, the method comprising orally administering to the subject a pharmaceutical composition comprising DGLA or a derivative thereof, 15-HETrE or a derivative thereof, or a combination thereof.

In a further aspect, the present disclosure provides methods of treating Peyronie's disease in a subject in need thereof, the method comprising administering to the subject a pharmaceutical composition comprising DGLA or a derivative thereof, 15-HETrE or a derivative thereof, or a combination thereof.

In yet another embodiment, the method further comprises reducing an amount of TGF-β in the subject in need thereof.

In still a further aspect, the present disclosure provides methods of treating cancer in a subject in need thereof, the method comprising administering to the subject a pharmaceutical composition comprising DGLA or a derivative thereof, 15-HETrE or a derivative thereof, or a combination thereof.

In still yet another embodiment, cancer is selected from the group consisting of renal cell carcinoma, hepatocellular carcinoma, cholangiocarcinoma, breast cancer, and cutaneous squamous cell carcinoma. In yet another embodiment, the method further comprises reducing an amount of TGF-β and/or EGFR in the subject in need thereof.

In yet another embodiment, the method further comprises reducing an amount of TGF-β and/or EGFR in the subject in need thereof.

In yet a further aspect, the present disclosure provides methods of treating a disease, a disorder, or a condition associated with T cell activation and/or mediated by CD40 in a subject in need thereof, the method comprising administering to the subject a pharmaceutical composition comprising DGLA or a derivative thereof, 15-HETrE or a derivative thereof, or a combination thereof.

In one embodiment, the disease, disorder, or condition associated with T cell activation and/or mediated by CD40 is selected from the group consisting of multiple sclerosis, inflammatory bowel disease, hematologic malignancy, breast cancer, and immunosuppression. In another embodiment, the inflammatory bowel disease is Crohn's disease or ulcerative colitis. In still yet another embodiment, the hematologic malignancy is selected from the group consisting of Hodgkin's lymphoma, non-Hodgkin's lymphoma, B-cell lymphomas, lymphocytic leukemia, multiple myeloma, and acute myeloid leukemia. In still a further embodiment, the immunosuppression is further associated with an organ transplant. In yet another embodiment, the method further comprises reducing an amount of one or more cytokines and/or chemokines in the subject in need thereof. In still another embodiment, the one or more cytokines and/or chemokines are selected from the group consisting of interleukin 15 receptor α (IL-15RA), interleukin 2 (IL-2), interleukin 2 receptor β (IL-2Rβ), interleukin 7 (IL-7), chemokine ligand 25 (CCL-25), cluster of differentiation 6 (CD6), signaling lymphocytic activation molecule family member 1 (SLAMF1), cluster of differentiation 224 (CD244), and activated leukocyte cell adhesion molecule (ALCAM). In yet another embodiment, T cell activation further comprises activation of one or more subtypes of T cells selected from the group consisting of Th1 T cells, Th17 T cells and Th2 T cells. In an additional embodiment, the T cell subtype is Th1 T cells and the one or more cytokines and/or chemokines are selected from the group consisting of chemokine ligand 16 (CCL-16), chemokine ligand 3 (CCL-3), chemokine C-X-C motif ligand 9 (CXCL-9), interleukin 18 (IL-18), interleukin 18 receptor 1 (IL-18R1), interleukin 18 binding protein (IL-18BP), chemokine ligand 4 (CCL-4), and chemokine ligand 3 (CCL-3). In yet an additional embodiment, the T cell subtype is Th17 T cells and the one or more cytokines and/or chemokines are selected from the group consisting of peptidase inhibitor 3 (PI3), CUB domain-containing protein 1 (CDCP1), interleukin 17α (IL-17α), interleukin 17 D (IL-17D), interleukin 17β (IL-17β), interleukin 12β (IL-12β), interleukin 17 receptor α (IL-17Rα), interleukin 20 (IL-20), chemokine C-X-C motif ligand 1 (CXCL1), and interleukin 12 (IL-12). In still an additional embodiment, the T cell subtype is Th2 T cells and the one or more cytokines and/or chemokines are selected from the group consisting of interleukin 5 receptor α (IL-5Rα), interleukin 10 (IL-10), interleukin 10 receptor β (IL-10Rβ), and interleukin 13 (IL-13). In a further embodiment, the T cell subtype is Th2 T cells and the one or more cytokines and/or chemokines are further selected from the group consisting of chemokine ligand 11 (CCL-11), chemokine ligand 23 (CCL-23), chemokine ligand 24 (CCL-24), chemokine ligand 28 (CCL-28), interleukin 24 (IL-24), and VEGF-A.

In still yet a further aspect, the present disclosure provides methods of inducing immunosuppression in a subject in need thereof, the method comprising administering to the subject a pharmaceutical composition comprising DGLA or a derivative thereof, 15-HETrE or a derivative thereof, or a combination thereof.

In one embodiment, the subject has received or will receive a renal transplant.

In an even further aspect, the present disclosure provides methods of treating scleroderma in a subject in need thereof, the method comprising administering to the subject a pharmaceutical composition comprising DGLA or a derivative thereof, 15-HETrE or a derivative thereof, or a combination thereof.

In one embodiment, the pharmaceutical composition comprises 15-HETrE or a derivative thereof, or DGLA or a derivative thereof. In one embodiment, the method includes reducing an amount of one or more cytokines and/or chemokines in the subject in need thereof. In one embodiment, the one or more cytokines and/or chemokines are selected from the group consisting of TGF-β, EGFR, VEGF-A, PDGF, and AXL.

In yet a further aspect, the present disclosure provides methods of treating a fibrosis disease or disorder in a subject in need thereof, the method comprising administering to the subject a pharmaceutical composition comprising DGLA or a derivative thereof, 15-HETrE or a derivative thereof, or a combination thereof.

In one embodiment, the fibrosis disease or disorder is selected from the group consisting of systemic fibrosis, renal fibrosis, lung fibrosis, skin fibrosis, myelofibrosis, and cardiac fibrosis. In one embodiment, the systemic fibrosis is radiation fibrosis. In one embodiment, the systemic fibrosis is radiation fibrosis. In one embodiment, the renal fibrosis is glomerular diseases, tubulointerstitial disease, iatrogenic nephropathy, and/or renal ischemia. In one embodiment, the glomerular diseases include but are not limited to focal segmental glomerulosclerosis, IgA nephropathy, crescentic glomerulonephritis, lupus nephritis, and diabetic nephropathy. In one embodiment, lung fibrosis is interstitial lung disease. In one embodiment, lung fibrosis further comprises idiopathic pulmonary fibrosis, scleroderma, radiation fibrosis, iatrogenic, sarcoidosis, mixed connective tissue disease, polymyositis, dermatomyositis, and/or systemic lupus erythematosus. In one embodiment, skin fibrosis further comprises scleroderma, systemic sclerosis, nephrogenic fibrosing dermopathy, mixed connective tissue disease, scleromyxedema, scleredema, eosinophilic fasciitis, and/or iatrogenic fibrosis. In one embodiment, the method includes reducing an amount of one or more cytokines and/or chemokines in the subject in need thereof. In one embodiment, the one or more cytokines and/or chemokines are selected from the group consisting of TGF-β, PDGF, EGFR, VEGF-A, and/or AXL. In one embodiment, the one or more cytokines and/or chemokines are TGF-β and PDGF. In one embodiment, the one or more cytokines and/or chemokines are TGF-β, PDGF, VEGF-A, and EGFR. In one embodiment, the one or more cytokines and/or chemokines are TGF-β, PDGF, and VEGF-A. In one embodiment, the one or more cytokines and/or chemokines is TGF-β.

In one embodiment, the composition is administered to the subject in an amount sufficient to provide up to about 8 g of DGLA or a derivative thereof per day. In one embodiment, the composition is administered to the subject in an amount sufficient to provide about 4 g to about 8 g of DGLA or a derivative thereof per day.

In one embodiment, the composition is administered to the subject in an amount sufficient to provide up to about 8 g of 15-HETrE or a derivative thereof per day. In one embodiment, the composition is administered to the subject in an amount sufficient to provide about 4 g to about 8 g of 15-HETrE or a derivative thereof per day.

In one embodiment, the composition is administered to the subject in an amount sufficient to provide up to about 8 g of DGLA or a derivative thereof per day and up to about 8 g of 15-HETrE or a derivative thereof per day. In one embodiment, the composition is administered to the subject in an amount sufficient to provide about 4 g to about 8 g of DGLA or a derivative thereof per day and about 4 g to about 8 g of 15-HETrE or a derivative thereof per day. In one embodiment, the composition is administered to the subject in an amount sufficient to provide about 500 mg to about 4 g of 15-HETrE or a derivative thereof per day and about 500 mg to about 4 g of DGLA or a derivative thereof per day.

In one embodiment, the composition is administered in 1 to 8 capsules per day. In one embodiment, the composition is administered in 1 to 4 capsules per day. In one embodiment, the composition is administered in 4 to 8 capsules per day.

In one embodiment, each capsule comprises about 200 mg to about 1 g of DGLA or a derivative thereof and/or 15-HETrE or a derivative thereof.

In one embodiment, the composition is administered to the subject for a period of at least about 1 week, at least about 2 weeks, at least about 4 weeks, at least about 6 weeks, at least about 8 weeks, or at least about 10 weeks.

In one embodiment, the composition is not encapsulated.

In one embodiment, the composition is administered by oral administration or by topical administration.

These and other embodiments of the invention are described in further detail below.

### DETAILED DESCRIPTION

While the present invention is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations (e.g., +/−10%) from a stated value can be used to achieve substantially the same results as the stated value. Also, the disclosure of ranges is intended as a continuous range including every value between the minimum and maximum values recited, as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein; and, in all instances, such ratios, ranges, and ranges of ratios represent various embodiments of the present invention.

### Compositions

In various embodiments, the present disclosure provides orally and/or topically deliverable pharmaceutical compositions comprising DGLA or a derivative thereof, 15-HETrE or a derivative thereof, or a combination thereof. The term DGLA herein refers to DGLA in free acid form. Compositions of the invention may also comprise a DGLA derivative in addition to or instead of DGLA. Such derivatives include alkyl esters; lower alky esters, such as DGLA methyl or ethyl ester; or DGLA in triglyceride form. In one embodiment, the present disclosure provides a pharmaceutical composition comprising DGLA or derivative thereof encapsulated in a capsule shell. In one embodiment, the composition is administered to the subject in an amount sufficient to provide up to about 8 g of DGLA or a derivative thereof per day. In one embodiment, the composition is administered to the subject in an amount sufficient to provide about 4 g to about 8 g of DGLA or a derivative thereof per day. In one embodiment, about 500 mg to about 1 g of DGLA or derivative thereof is encapsulated in the capsule shell.

15-Hydroxy-eicosa-8(Z),11(Z),13(E)-trienoic acid (“15-HETrE”) is a derivative of DGLA. As used herein, the term “15-HETrE” refers to 15-HETrE in its free acid form (e.g., 15-hydroxy-eicosa-8(Z),11(Z),13(E)-trienoic acid) and/or a pharmaceutically acceptable ester, derivative, conjugate or salt thereof; or mixtures of any of the foregoing. Compositions of the invention may also comprise a 15-HETrE derivative in addition to or instead of 15-HETrE. Such derivatives include alkyl esters; lower alky esters, such as 15-HETrE methyl or ethyl ester; or 15-HETrE in triglyceride form. In one embodiment, the present disclosure provides a pharmaceutical composition comprising 15-HETrE or derivative thereof encapsulated in a capsule shell. In one embodiment, the composition is administered to the subject in an amount sufficient to provide up to about 8 g of 15-HETrE or a derivative thereof per day. In one embodiment, the composition is administered to the subject in an amount sufficient to provide about 4 g to about 8 g of 15-HETrE or a derivative thereof per day. In one embodiment, about 500 mg to about 1 g of 15-HETRE or derivative thereof is encapsulated in the capsule shell.

In one embodiment, the composition is administered to the subject in an amount sufficient to provide up to about 8 g of DGLA or a derivative thereof per day and up to about 8 g of 15-HETrE or a derivative thereof per day. In one embodiment, the composition is administered to the subject in an amount sufficient to provide about 4 g to about 8 g of DGLA or a derivative thereof per day and about 4 g to about 8 g of 15-HETrE or a derivative thereof per day. In one embodiment, the composition is administered to the subject in an amount sufficient to provide about 500 mg to about 4 g of 15-HETrE or a derivative thereof per day and about 500 mg to about 4 g of DGLA or a derivative thereof per day.

In one embodiment, the capsule shell comprises gelatin (for example, Gelatin RXL or lime bone gelatin with a lower molecular weight). In another embodiment, the capsule shell comprises Gelatin RXL that has been treated by proteolytic enzyme to cut the gelatin pattern and effectively decrease its molecular weight. In another embodiment, the pharmaceutical composition comprises DGLA esters of D-Sorbitol and 1,4-sorbitan. In one embodiment, the capsule shell comprises (a) gelatin and (b) plasticizers selected from one or more of d-sorbitol and 1,4-sorbitans. In one embodiment, the gelatin is as described in U.S. Pat. No. 7,485,323, and is hereby incorporated by reference herein in its entirety.

In one embodiment, the plasticizer comprises 1,4-sorbitans in an amount from 20%-30%, for example, about 24% and 28% (on a dry basis), and a D-sorbitol content of about 30%-50%, for example, about 35% to 45% (on a dry basis).

In some embodiments, the capsule shell further comprises glycerol, purified water, titanium dioxide, medium chain triglycerides and lecithin.

In various embodiments, DGLA or derivative thereof and/or 15-HETrE or derivative thereof is present in a composition of the invention in an amount of about 50 mg to about 5000 mg, about 75 mg to about 2500 mg, or about 100 mg to about 1000 mg, for example, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg. In any such embodiment, the composition can further comprise DGLA or derivative thereof and/or 15-HETrE esters of D-Sorbitol and 1,4-sorbitan or derivative thereof.

A pharmaceutical composition of the invention comprises a therapeutically effective amount of a lysine salt of 15-HETrE or derivative thereof. The salt form of 15-HETrE or derivative thereof may be the sole significant active ingredient in that composition and in the methods and uses as stated herein. The salt form of 15-HETrE or derivative thereof may be the sole active ingredient. Alternatively, the salt form of 15-HETrE or derivative thereof may be combined for co-formulation or co-administration with other agents for treating a disease or disorder. If an additional active agent is to be used, the salt form of 15-HETrE or derivative thereof can be co-formulated as a single dosage unit or can be formulated as two to a plurality of dosage units for coordinated, combination or concomitant administration.

In one embodiment, the pharmaceutical composition comprises at least about 50%, at least about 60%, at least about 70%, at least about 80% or at least about 90%, by weight of the salt form of 15-HETrE or derivative thereof.

In another embodiment, the salt form of 15-HETrE or derivative thereof is present in a composition of the invention in an amount of about 1 mg to about 10,000 mg, about 25 mg to about 7500 mg, about 25 mg to about 5000 mg, about 50 mg to about 5000 mg, about 50 mg to about 3000 mg, about 75 mg to about 2500 mg, or about 100 mg to about 1000 mg, for example about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In one embodiment, the salt form of 15-HETrE or derivative thereof present in a composition of the invention comprises at least 90% by weight of the salt form of 15-HETrE or derivative thereof. Compositions containing the salt form of 15-HETrE or derivative thereof can comprise even higher purity, for example at least 91% by weight, at least 92% by weight, at least 93% by weight, at least 94% by weight, at least 95% by weight, at least 96% by weight or at least 97% by weight of the salt form of 15-HETrE or derivative thereof.

In one embodiment, a composition of the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of total fatty acids, of fatty acids other than DGLA or derivative thereof and/or 15-HETrE or derivative thereof.

In another embodiment, DGLA or derivative thereof and/or 15-HETrE or derivative thereof represents at least about 30%, about 40%, about 50%, at least about 60%, at least about 70%, at least about 80%, at least about 90%, at least about 95%, at least about 97%, at least about 98%, at least about 99%, or 100%, by weight, of all fatty acids present in a composition of the invention.

In one embodiment, a composition of the invention when placed in a standard disintegration test, for example, as set forth in USP 2040 (Disintegration and Dissolution of Dietary Supplements) with water as the Medium has a DGLA or derivative thereof and/or 15-HETrE or derivative thereof release rate less than about 60 minutes, less than about 50 minutes, less than about 40 minutes, less than about 30 minutes, or less than 20 minutes after storage for about 1 month, about 2 months, or about 3 months at 40° C./75% RH.

In one embodiment, after storage for about 1 month, about 2 months, about 3 months, or about 6 months at 40° C./75% RH, a composition of the invention comprises less than about 5% DGLA esters and/or 15-HETrE esters by weight of all fatty acids, less than about 4% DGLA esters and/or 15-HETrE esters by weight of all fatty acids, less than about 3% DGLA esters and/or 15-HETrE esters by weight of all fatty acids, less than about 2% DGLA esters and/or 15-HETrE esters by weight of all fatty acids, or less than about 1% DGLA esters and/or 15-HETrE esters by weight of all fatty acids.

In some embodiments, the pharmaceutical composition is in a form suitable for topical administration. In various embodiments, the invention provides pharmaceutical compositions, for example topically deliverable compositions, comprising DGLA or derivative thereof and/or 15-HETrE or derivative thereof. In yet another embodiment, present disclosure provides topical pharmaceutical compositions comprising, for example, an amount (e.g., a therapeutically effective amount) of DGLA or derivative thereof and/or 15-HETrE or derivative thereof.

### Methods

Any composition of the invention, including compositions described herein above or compositions that can for formulated from combining various embodiments of the present disclosure, can be used in treatment or prevention of: skin disorders and diseases, including acne vulgaris, acne rosacea, atopic dermatitis, psoriasis, pruritus/itch, radiation protection, dry skin, smooth skin, healthy skin, anti-aging, and photoprotection; urinary disorders and diseases including bladder cancer, cystocele, hematuria, interstitial cystitis, neurogenic bladder, Peyronie's disease, prostate disease, incontinence, urinary tract infection, and vasicoureteral reflux; renal disease and disorders including kidney failure, acute kidney injury, chronic kidney disease, and polycystic kidney disease; rheumatic disease including ankylosing spondylitis, fibromyalgia, gout, infectious arthritis, lupus, osteoarthritis, polymyalgia rheumatica, psoriatic arthritis, reactive arthritis, rheumatoid arthritis, sclerodoma; respiratory disorders including inflammatory lung disease, respiratory tract infections, pleural cavity disease, pulmonary vascular disease, pneumonia, pulmonary embolism, and lung cancer; and cardiovascular disorders including acute cardiac ischemic events, acute myocardial infarction, angina, arrhythmia, atrial fibrulation, atherosclerosis, arterial fibrillation, cardiac insufficiency, cardiovascular disease, chronic heart failure, chronic stable angina, congestive heart failure, coronary artery disease, coronary heart disease, deep vein thrombosis, diabetes, diabetes mellitus, diabetic neuropathy, diastolic dysfunction in subjects with diabetes mellitus, edema, essential hypertension, eventual pulmonary embolism, fatty liver disease, heart disease, heart failure, homozygous familial hypercholesterolemia (HoFH), homozygous familial sitosterolemia, hypercholesterolemia, hyperlipidemia, hypertension, hypertriglyceridemia, metabolic syndrome, mixed dyslipidemia, moderate to mild heart failure, myocardial infarction, obesity management, paroxysmal atrial/arterial fibrillation/fibrulation/flutter, paroxysmal supraventricular tachycardias (PSVT), particularly severe or rapid onset edema, platelet aggregation, primary hypercholesterolemia, primary hyperlipidemia, pulmonary arterial hypertension, pulmonary hypertension, recurrent hemodynamically unstable ventricular tachycardia (VT), recurrent ventricular arrhythmias, recurrent ventricular fibrillation (VF), ruptured aneurysm, sitisterolemia, stroke, supraventricular tachycardia, symptomatic atrial fibrillation/flutter, tachycardia, type-II diabetes, vascular disease, venous thromboembolism, ventricular arrhythmias, and other cardiovascular events, and Alzheimer's disease.

In some embodiments, compositions of the invention, such as DGLA or derivative thereof and/or 15-HETrE or derivative thereof, can reduce (i.e., downregulate) expression of one or more makers associated with fibrotic diseases or disorders and cancers. In various embodiments, in response to administration of a composition comprising DGLA or derivative thereof and/or 15-HETrE or derivative thereof, expression of one or more markers associated with fibrosis and/or proliferation is reduced in the subject. In some embodiments, the markers are markers expressed in high levels in multiple fibrotic diseases, malignancies, and other diseases with fibrotic elements (e.g. multiple sclerosis). Examples of markers associated with fibrotic disease and cancer include transforming growth factor beta (TGF-β), epidermal growth factor receptor (EGFR), hepatocyte growth factor (HGF), platelet derived growth factor subunit A (PDGF subunit A), vascular endothelial growth factor (VEGF-A), tyrosine-protein kinase receptor UFO (AXL), and transforming growth factor alpha (TGF-α). Non-limiting examples of fibrotic diseases include systemic fibrosis (i.e., radiation fibrosis), liver fibrosis and/or cirrhosis, renal fibrosis, lung fibrosis and/or interstitial lung disease, skin fibrosis, cardiac fibrosis, ocular fibrosis, ocular disease, connective tissue disorders, myelofibrosis, cancers, and other related fibrotic diseases.

In some embodiments, the subject experiences a reduction in one or more markers associated with other diseases associated with liver fibrosis and/or cirrhosis, and or treatment or prevention of liver fibrosis and/or cirrhosis. Non-limiting examples of liver fibrosis and/or cirrhosis related diseases include non-alcoholic fatty liver disease (NAFLD), alcoholic hepatitis, primary sclerosing cholangitis, primary biliary cholangitis, viral hepatitis (Chronic Hepatitis C, HBV), autoimmune hepatitis, Iatrogenic and/or drug induced liver injury, and/or hepatic veno-occlusive disease. In some embodiments, the subject experiences a reduction in one or more markers associated with NAFLD. Examples of markers associated with NAFLD include TGF-β, HGF, and PDGF. In another embodiment, the subject experiences a reduction in one or more markers associated with alcoholic hepatitis. Examples of markers associated with alcoholic hepatitis include TGF-β and AXL. In yet another embodiment, the subject experiences a reduction in one or more markers associated with primary sclerosing cholangitis. Examples of markers associated with primary sclerosing cholangitis include TGF-β and EGFR. In some embodiments, the subject experiences a reduction in one or more markers associated with primary biliary cholangitis. Examples of markers associated with primary biliary cholangitis include TGF-β and EGFR. In yet another embodiment, the subject experiences a reduction in one or more markers associated with viral hepatitis. Examples of markers associated with viral hepatitis include TGF-β and AXL.

In some embodiments, the subject experiences a reduction in one or more markers associated with autoimmune hepatitis. An example of a marker associated with autoimmune hepatitis is TGF-β.

In another embodiment, the subject experiences a reduction in one or more markers associated with iatrogenic and/or drug induced liver injury. An example of marker associated with latrogenic and/or drug induced liver injury is TGF-β.

In some embodiments, the subject experiences a reduction in one or more markers associated hepatic veno-occlusive disease. Examples of markers associated with veno-occlusive disease TGF-β and VEGF-A. Non-limiting examples of renal fibrosis include glomerular diseases, tubulointerstitial disease, and latrogenic nephropathy/renal ischemic.

In some embodiments, the subject experiences a reduction in one or more markers associated with glomerular diseases, and/or treatment or prevention of one or more glomerular diseases. Examples of markers associated with glomerular diseases include TGF-β, EGFR, PDGF, and VEGF-A. Non-limiting examples of glomerular diseases include focal segmental glomerulosclerosis (FSGS), IgA nephropathy, crescentic glomerulonephritis, lupus nephritis, and diabetic nephropathy. In another embodiment, the subject experiences a reduction in one or more markers associated with tubulointerstitial disease. Examples of markers associated with tubulointerstitial disease include TGF-β, EGFR, PDGF, and VEGF-A. In some embodiments, the subject experiences a reduction in one or more markers associated with latrogenic nephropathy/renal ischemia. Examples of markers associated with latrogenic nephropathy/renal ischemia include TGF-β, EGFR, PDGF, and VEGF-A.

In some embodiments, the subject experiences a reduction in one or more markers associated with systematic fibrosis, and/or treatment or prevention of systemic fibrosis. Examples of markers associated with systematic fibrosis include TGF-β and PDGF subunit A.

In yet another embodiment, the subject experiences a reduction in one or more markers associated with liver fibrosis and/or cirrhosis, and/or treatment or prevention of liver fibrosis and/or cirrhosis. Examples of markers associated with liver fibrosis and/or cirrhosis include TGF-β, TGF-α, HGF, PDGF subunit A, and AXL.

In another embodiment, the subject experiences a reduction in one or more markers associated with renal fibrosis, and/or treatment or prevention of renal fibrosis. Examples of markers associated with renal fibrosis include TGF-β, EGFR, PDGF, and VEGF-A.

In various embodiments, the subject experiences a reduction in one or more markers associated with lung fibrosis and/or interstitial lung disease, and/or treatment or prevention of lung fibrosis and/or interstitial lung disease. Examples of markers associated with lung fibrosis and/or interstitial lung disease include TGF-β, EGFR, AXL, PDGF, and VEGF-A.

In another embodiment, the subject experiences a reduction in one or more markers associated with skin fibrosis, and/or treatment or prevention of skin fibrosis. Examples of markers associated with skin fibrosis include TGF-β, PDGF, and VEGF-A.

In some embodiments, the subject experiences a reduction in one or more markers associated with cardiac fibrosis, and/or treatment or prevention of cardiac fibrosis. Examples of markers associated with cardiac fibrosis include TGF-β, EGFR, and PDGF.

In some embodiments, the subject experiences a reduction in one or more markers associated with ocular diseases, and/or treatment or prevention of ocular diseases. Non-limiting examples of ocular diseases include corneal opacification, glaucoma, age-related macular degeneration (AMD), cataract, and diabetic retinopathy (DR). Examples of markers associated with ocular diseases include TGF-β and VEGF-A.

In another embodiment, the subject experiences a reduction in one or more markers associated with connective tissue disorders, and/or treatment or prevention of connective tissue disorders. In some embodiments, the subject experiences a reduction in one or more markers associated with myelofibrosis. An example of a markers associated with myelofibrosis is TGF-β.

In another embodiment, the subject experiences a reduction in one or more markers associated with Alzheimer's disease, and/or treatment or prevention of Alzheimer's disease. An example marker associated with Alzheimer's disease includes TGF-β.

In another embodiment, the subject experiences a reduction in one or more markers associated with Peyronie's disease, and/or treatment or prevention of Alzheimer's disease. An example marker associated with Peyronie's disease includes TGF-β.

In some embodiments, the subject experiences a reduction in one or more lung diseases, such as lung fibrosis and/or interstitial lung disease, and/or treatment or prevention of lung fibrosis and/or interstitial lung disease. Non-limiting examples of lung diseases, such as lung fibrosis and/or interstitial lung disease include idiopathic pulmonary fibrosis, sarcoidosis, mixed connective tissue disease, polymyositis, dermatomyositis, and systemic lupus erythematosus. Non-limiting examples of markers associated with skin diseases and disorders include TGF-β, EGFR, VEGF-A, PDGF, and AXL.

In some embodiments, the subject experiences a reduction in one or more skin diseases or disorders, and/or treatment or prevention of one or more skin diseases or disorders. Non-limiting examples of skin diseases and disorders include scleroderma/systemic sclerosis, nephrogenic fibrosing dermopathy, mixed connective tissue disease, scleromyxedema, scleredema, eosinophilic fasciitis, iatrogenic fibrosis, scleroderma, and radiation fibrosis. Non-limiting examples of markers associated with skin diseases and disorders include TGF-β, EGFR, VEGF-A, PDGF, and AXL.

In some embodiments, the subject experiences a reduction in one or more markers associated with cardiac disease, and/or treatment or prevention of one or more cardiac diseases. Examples of markers associated with cardiac disease include TGF-β, EGFR, and PDGF.

In some embodiments, the subject experiences a reduction in one or more connective tissue disorders, and/or treatment or prevention of one or more connective tissue disorders. Non-limiting examples of connective tissue disorders include Marfan's syndrome, Loeys-Dietz syndrome, and vascular Ehlers-Danlos syndrome (EDS). In some embodiments, the subject experiences a reduction in on or more markers associated with Marfan's syndrome. An example of a marker associated with Marfan's syndrome is TGF-β. In another embodiment, the subject experiences a reduction in one or more markers associated with Loeys-Dietz syndrome. An example of a marker associated with Loeys-Dietz syndrome is TGF-β. In yet another embodiment, the subject experiences a reduction in one or more markers associated with vascular EDS. An example of a marker associated with vascular EDS is TGF-β.

In another embodiment, the subject experiences treatment or prevention of scleroderma. In some embodiments, the subject experiences a reduction in one or more markers associated with scleroderma. Examples of markers associated with scleroderma include TGF-β, EGFR, VEGF-A, PDGF, and AXL.

In another embodiment, the subject experiences treatment or prevention of one or more fibrosis diseases or disorders. In some embodiments, the subject experiences a reduction in one or more markers associated with fibrosis diseases or disorders. Non-limiting examples of fibrosis diseases or disorders include systemic fibrosis, renal fibrosis, lung fibrosis, skin fibrosis, myelofibrosis, and cardiac fibrosis. In one embodiment, the systemic fibrosis is radiation fibrosis. In one embodiment, the systemic fibrosis is radiation fibrosis. In one embodiment, the renal fibrosis is glomerular diseases, tubulointerstitial disease, iatrogenic nephropathy, and/or renal ischemia. In one embodiment, the glomerular diseases include but are not limited to focal segmental glomerulosclerosis, IgA nephropathy, crescentic glomerulonephritis, lupus nephritis, and diabetic nephropathy. In one embodiment, lung fibrosis is interstitial lung disease. In one embodiment, lung fibrosis further comprises idiopathic pulmonary fibrosis, scleroderma, radiation fibrosis, iatrogenic, sarcoidosis, mixed connective tissue disease, polymyositis, dermatomyositis, and/or systemic lupus erythematosus. In one embodiment, skin fibrosis further comprises scleroderma, systemic sclerosis, nephrogenic fibrosing dermopathy, mixed connective tissue disease, scleromyxedema, scleredema, eosinophilic fasciitis, and/or iatrogenic fibrosis. In one embodiment, the subject experiences a reduction in an amount of one or more cytokines and/or chemokines in the subject in need thereof. Examples of markers associated with fibrosis diseases or disorders include TGF-β, PDGF, EGFR, VEGF-A, and/or AXL.

In another embodiment, the subject experiences treatment or prevention of one or more cancers. In some embodiments, the subject experiences a reduction in one or more markers associated with cancer. Non-limiting examples of cancer include renal cell carcinoma, hepatocellular carcinoma, cholangiocarcinoma, breast cancer, and cutaneous squamous cell carcinoma. In some embodiments, the subject experiences a reduction in one or more markers associated with renal cell carcinoma. Examples of markers associated with renal cell carcinoma include TGF-β and EGFR. In some embodiments, the subject experiences a reduction in one or more markers associated with hepatocellular carcinoma. Examples of markers associated with hepatocellular carcinoma include TGF-β and EGFR. In some embodiments, the subject experiences a reduction in one or more markers associated with cholangiocarcinoma. Examples of markers associated with cholangiocarcinoma include TGF-β and EGFR. In some embodiments, the subject experiences a reduction in one or more markers associated with breast cancer. Examples of markers associated with breast cancer include TGF-β and EGFR. In some embodiments, the subject experiences a reduction in one or more markers associated with cutaneous squamous cell carcinoma. Examples of markers associated with cutaneous squamous cell carcinoma include TGF-β and EGFR.

In some embodiments, compositions of the invention, such as compositions comprising DGLA or a derivative thereof and/or 15-HETrE or a derivative thereof, can induce immunosuppression. In various embodiments, immunosuppression is induced in an organ transplantation subject, such as a subject who has not yet received an organ transplant, who is receiving an organ transplant, or who has received an organ transplant. In some embodiments, the organ transplant is a renal transplant.

In some embodiments, compositions of the invention, such as compositions comprising DGLA or a derivative thereof and/or 15-HETrE or a derivative thereof, can reduce (i.e., downregulate) one or more makers associated with inflammatory conditions or T-cell activation. In various embodiments, in response to administration of a composition comprising DGLA or a derivative thereof and/or 15-HETrE or a derivative thereof, the subject experiences a reversal of systemic immune activation. In another embodiment, the subject experiences a reduction in one or more markers associated inflammatory cytokines and chemokines. In some embodiments, the associated inflammatory cytokines and chemokines are from multiple immune axes. Non-limiting examples of markers associated cytokines and chemokines include T-cell and natural killer cell activation factors, T-helper (Th) 1, Th2, Th17, Th22, and T-regulated axes, general immune, and inflammation. In some embodiments, the T-cell activation factor, CD40 is associated with the inflammatory condition. Non-limiting examples of inflammatory diseases associated with CD40 include atopic dermatitis, T-cell mediated autoimmune diseases, hematologic malignancies, solid organ tumors such as breast carcinoma, and atherosclerosis. Examples of T-cell medicated autoimmune diseases include multiple sclerosis, rheumatoid arthritis, type 1 diabetes mellitus, and inflammatory bowel diseases. In various embodiments, in response to administration of a composition comprising DGLA and/or 15-HETrE, the subject experiences immunosuppression. In some embodiments, the immunosuppression is induced by the composition. Non-limiting examples of indications involving immunosuppression include transplantation, such as organ transplantation (e.g., kidney/renal). Examples of inflammatory bowel diseases include Crohn's disease, systemic lupus erythematosus, lupus nephritis, psoriasis, acne, asthma, and hidradenitis suppurative. Non-limiting examples of hematologic malignancies include Hodgkin's lymphoma, Non-Hodgkin's lymphoma, b-cell lymphomas, lymphocytic leukemia, multiple myeloma, and acute myeloid leukemia.

In various embodiments, in response to administration of a composition comprising DGLA and/or 15-HETrE, the subject experiences a reduction in one or more of the following makers: general inflammation, innate immunity, T-Cell/natural killer (NK) cell activation, Th1, Th17/Th1, Th2, Th2 and other chemokines and adhesion molecules, and fibrotic and proliferative. For example, general inflammation general inflammation markers include but are not limited to tumor necrosis factor related apoptosis-inducing ligand (TRAIL), interleukin 1 receptor type 2 (IL.1RT2), interleukin 1 receptor type 1/interleukin 1 receptor type I (IL.RT1/1L1R1), matrix metalloproteinase-1 (MMP.1), matrix metalloproteinase-10 (MMP.10), matrix metalloproteinase-7 (MMP7), and tumor necrosis factor receptor superfamily/cluster of differentiation 137 (TNFRSF/CD137). For example, innate immunity related markers include but are not limited to cluster of differentiation 163 (CD163), interleukin 6 receptor alpha (IL.6RA), granulin gene (GRN), macrophage receptor (MARCO), peptidoglycan recognition protein 1 (PGLYRP1), and colony-stimulating factor-1 (CSF.1). For example, T cell/NK cell activation markers include but are not limited to interleukin 15 receptor subunit alpha (IL.15RA), interleukin 2 (IL2), interleukin 2 receptor subunit beta (IL.2RB), C-C motif chemokine ligand 25 (CCL-25), interleukin 7 (IL7), CD6, signaling lymphocytic activation molecule 1 (SLAMF1), cluster of differentiation 224 (CD244), cluster of differentiation 40 (CD40), and activated leukocyte cell adhesion molecule (ALCAM). For example, Th1 related makers include but are not limited to C-C motif chemokine ligand 16 (CCL-16), C-C motif chemokine ligand 3 (CCL-3), C-X-C motif chemokine ligand 9 (CXCL9), interleukin 18 (IL18), interleukin 18 receptor 1 (IL.18R1), interleukin 18 binding protein (IL.18BP), C-C motif chemokine ligand 4 (CCL-4), and C-C motif chemokine ligand 3/microphage inflammatory protein 1 alpha (CCL-3/MIP-1-Alpha). For example, Th17/Th1 related makers include but are not limited to peptidase inhibitor 3/elafin (PI3/elafin), cub domain-containing protein/cluster domain of differentiation 318 (CDCP1/CD318), interleukin 17A (IL.17A), interleukin 17D (IL.17D), interleukin 12B (IL.12B), interleukin 17 receptor alpha (IL.17RA), interleukin 20 (IL.20), C-X-C motif chemokine ligand 1 (CXCL1), and interleukin 12 (IL12). For example, Th2 related markers include but are not limited to interleukin 5 receptor alpha (IL.5Rα), interleukin 10 (IL10), interleukin-10 receptor subunit beta (IL.10RB), and interleukin 13 (IL13). For example, Th2 and other related chemokines and adhesion molecules include but are not limited to C-C motif chemokine ligand 23 (CCL-23), C-C motif chemokine ligand 24 (CCL-24), C-C motif chemokine ligand 28 (CCL-28), interleukin 24 (IL.24), C-C motif chemokine ligand 11 (CCL-11), and VEGF-A.

The term “treatment” in relation a given disease or disorder includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder.

The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In one embodiment, the subject is determined to have a low baseline eosinophil count as compared to a reference level. In one embodiment, the subject is determined to have a low baseline eosinophil count prior to administration of the DGLA.

The term “reference level” includes, but is not limited to, a level from a sample collected from a healthy patient. A reference level can also be determined from a plurality of samples collected from a population of healthy patients. As one example, a low eosinophil cell count can be determined based on an eosinophil cell count determined from a population of healthy patients, or a subset of healthy patients, for example, healthy patients of a particular ethnicity. In other embodiments, the reference level is a value determined from a sample collected at an earlier time point (e.g., 1 day, 3 days, 1 week, 1 month, 3 months, 6 months, 12 months, or more) from the same patient that is undergoing treatment. In some embodiments, the reference level may be based on values known by those of skill in the art or developed by a medical agency.

In various embodiments, compositions of the invention are administered in an amount sufficient to provide a daily DGLA or derivative thereof dose and/or a 15-HETrE or derivative thereof dose of about 50 mg to about 10000 mg, about 100 mg to about 7500 mg, or about 100 mg to about 5000 mg, for example, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg about 2600 mg, about 2700 mg, about 2800 mg, about 2900 mg, about 3000 mg, about 3100 mg, about 3200 mg, about 3300 mg, about 3400 mg, about 3500 mg, 3600 mg, about 3700 mg, about 3800 mg, about 3900 mg, about 4000 mg, about 4100 mg, about 4200 mg, about 4300 mg, about 4400 mg, about 4500 mg, 4600 mg, about 4700 mg, about 4800 mg, about 4900 mg, about 5000 mg, about 5100 mg, about 5200 mg, about 5300 mg, about 5400 mg, about 5500 mg of DGLA or derivative thereof and/or 15-HETrE or derivative thereof per day.

In one embodiment, the invention provides a method of treating atopic dermatitis, for example, mild to moderate atopic dermatitis. In one embodiment, the method comprises administering to a subject in need of such treatment DGLA or derivative thereof and/or 15-HETrE or derivative thereof in an amount of about 500 mg to about 3 g per day, about 1 g to about 2.5 g per day, about 1 g per day, or about 2 g per day. In one embodiment, the DGLA or derivative thereof and/or 15-HETrE or derivative thereof is administered to the subject daily for a period of at least about 2 weeks, at least about 4 weeks, or at least about 8 weeks. In a related embodiment, upon treatment in accordance with the present invention, for example, over a period of about 1 to about 12 weeks, about 1 to about 8 weeks, or about 1 to about 4 weeks, the subject or subject group exhibits one or more of the following outcomes:

(a) a reduction in eczema area and severity index (EASI) score relative to baseline or placebo control;

(b) a reduction in percentage of area of an anatomical site affected by atopic dermatitis relative to baseline or control;

(c) a reduction in investigator's global assessment score relative to baseline or placebo control;

(d) a reduction in intensity of erythema, edema/population, oozing/crusts, excoriation, lichenification and/or dryness relative to baseline or placebo control;

(e) a reduction in erythema, edema/population, oozing/crusts, excoriation, lichenification and/or dryness relative to baseline or placebo control;

(f) a reduction in body surface area (BSA) affected by atopic dermatitis relative to baseline or placebo control;

(g) a reduction in loss of sleep relative to baseline or placebo control;

(h) a reduction in occurrence of pruritis (itch) relative to baseline or placebo control;

(i) a reduction in severity of pruritis as an average of the prior three days and/or nights on a visual analog scale;

(j) a reduction in SCORAD score relative to baseline or placebo control;

(k) an improved patient-oriented Eczema Measure (POEM) compared to baseline or placebo control;

(l) a reduction in number of days in the preceding week in which the subject reported that the skin was itchy due to eczema;

(m) a reduction in number of days in the preceding week in which the subject reported that their sleep was disturbed due to their eczema;

(n) a reduction in number of days in the preceding week in which the subject experienced skin bleeding;

(o) a reduction in number of days in the preceding week in which the subject experienced skin weeping or oozing clear fluid;

(p) a reduction in number of days in the preceding week in which the subject's skin cracked;

(q) a reduction in number of days in the preceding week in which the subject's skin flaked;

(r) a reduction in number of days in the preceding week in which the subject experienced dry skin;

(s) an increase in trans epidermal water loss compared to baseline or placebo control;

(t) an increase in plasma total and free DGLA compared to baseline;

(u) an increase in DGLA:AA ratio compared to baseline or placebo control; and/or

(v) a reduction in arterial blood pressure compared to baseline or placebo control.

In one embodiment, methods of the present invention comprise measuring baseline levels of one or more markers or parameters set forth in (a)-(v) above prior to dosing the subject or subject group. In another embodiment, the methods comprise administering a composition as disclosed herein to the subject after baseline levels of one or more markers or parameters set forth in (a)-(v) are determined, and subsequently taking an additional measurement of said one or more markers.

In another embodiment, upon treatment with a composition of the present invention, for example, over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, or all 22 of outcomes (a)-(v) described immediately above.

In another embodiment, upon treatment with a composition of the present invention, the subject or subject group exhibits one or more of the following outcomes:

(a) a reduction in eczema area and severity index (EASI) score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(b) a reduction in percentage of area of an anatomical site affected by atopic dermatitis relative to baseline or control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(c) a reduction in investigator's global assessment score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(d) a reduction in intensity of erythema, edema/population, oozing/crusts, excoriation, lichenification and/or dryness relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(e) a reduction in erythema, edema/population, oozing/crusts, excoriation, lichenification and/or dryness relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(f) a reduction in body surface area (BSA) affected by atopic dermatitis relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(g) a reduction in loss of sleep relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(h) a reduction in occurrence of pruritis (itch) relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(i) a reduction in severity of pruritis as an average of the prior three days and/or nights on a visual analog scale of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(j) a reduction in SCORAD score relative to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(k) an improved patient-oriented Eczema Measure (POEM) compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(l) a reduction in number of days in the preceding week in which the subject reported that their skin was itchy due to eczema of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(m) a reduction in number of days in the preceding week in which the subject reported that their sleep was disturbed due to their eczema of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(n) a reduction in number of days in the preceding week in which the subject experienced skin bleeding of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(o) a reduction in number of days in the preceding week in which the subject experienced skin weeping or oozing clear fluid of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(p) a reduction in number of days in the preceding week in which the subject's skin cracked of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(q) a reduction in number of days in the preceding week in which the subject's skin flaked of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(r) a reduction in number of days in the preceding week in which the subject experienced dry skin of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(s) an increase in trans epidermal water loss compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(t) an increase in plasma total and free DGLA compared to baseline of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%;

(u) an increase in DGLA:AA ratio compared to baseline or placebo control of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%; and/or

(v) a reduction in mean arterial blood pressure of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95%.

In another embodiment, upon treatment with a composition of the present invention after a single dose administration or multiple dose administration, for example over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of or all 22 of outcomes (a)-(v) described immediately above.

In another embodiment, upon treatment with a composition of the present invention, the subject or subject group exhibits one or more of the following outcomes:

(a) a reduction in TGF-α levels relative to baseline, placebo control, and/or untreated patient;

(b) a reduction in TGF-β levels relative to baseline, placebo control, and/or untreated patient;

(c) a reduction in EGFR levels relative to baseline, placebo control, and/or untreated patient;

(d) a reduction in VEGF-A relative to baseline, placebo control, and/or untreated patient;

(e) a reduction in AXL levels relative to baseline, placebo control, and/or untreated patient;

(f) a reduction in IL-15RA levels relative to baseline, placebo control, and/or untreated patient;

(g) a reduction in IL-2Rβ levels relative to baseline, placebo control, and/or untreated patient;

(h) a reduction in IL-7 levels relative to baseline, placebo control, and/or untreated patient;

(i) a reduction in CCL-25 levels relative to baseline, placebo control, and/or untreated patient;

(j) a reduction in CD6 levels relative to baseline, placebo control, and/or untreated patient;

(k) a reduction in SLAMF1 level s relative to baseline, placebo control, and/or untreated patient;

(l) a reduction in CD244 levels relative to baseline, placebo control, and/or untreated patient;

(m) a reduction in ALCAM levels relative to baseline, placebo control, and/or untreated patient;

(n) a reduction in CCL-16 levels relative to baseline, placebo control, and/or untreated patient;

(o) a reduction in CCL-3 levels relative to baseline, placebo control, and/or untreated patient;

(p) a reduction in IL-2Rβ levels relative to baseline, placebo control, and/or untreated patient;

(q) a reduction in CXCL-9 levels relative to baseline, placebo control, and/or untreated patient;

(r) a reduction in IL-18 levels relative to baseline, placebo control, and/or untreated patient;

(s) a reduction in IL-18R1 levels relative to baseline, placebo control, and/or untreated patient;

(t) a reduction in IL-18BP levels relative to baseline, placebo control, and/or untreated patient;

(u) a reduction in CCL-4 levels relative to baseline, placebo control, and/or untreated patient;

(v) a reduction in CCL-3 levels relative to baseline, placebo control, and/or untreated patient;

(w) a reduction in PI3 levels relative to baseline, placebo control, and/or untreated patient;

(x) a reduction in CDCP1 levels relative to baseline, placebo control, and/or untreated patient;

(y) a reduction in IL-17α levels relative to baseline, placebo control, and/or untreated patient;

(z) a reduction in IL-17D levels relative to baseline, placebo control, and/or untreated patient;

(aa) a reduction in IL-17β levels relative to baseline, placebo control, and/or untreated patient;

(bb) a reduction in IL-12β levels relative to baseline, placebo control, and/or untreated patient;

(cc) a reduction in IL-17Rα levels relative to baseline, placebo control, and/or untreated patient;

(dd) a reduction in IL-20 levels relative to baseline, placebo control, and/or untreated patient;

(ee) a reduction in CXCL1 levels relative to baseline, placebo control, and/or untreated patient;

(ff) a reduction in IL-12 levels relative to baseline, placebo control, and/or untreated patient;

(gg) a reduction in IL-5Rα levels relative to baseline, placebo control, and/or untreated patient;

(hh) a reduction in IL-10 levels relative to baseline, placebo control, and/or untreated patient;

(ii) a reduction in IL-10Rβ levels relative to baseline, placebo control, and/or untreated patient;

(jj) a reduction in IL-13 levels relative to baseline, placebo control, and/or untreated patient;

(kk) a reduction in CCL-11 levels relative to baseline, placebo control, and/or untreated patient;

(ll) a reduction in CCL-23 levels relative to baseline, placebo control, and/or untreated patient;

(mm) a reduction in CCL-24 levels relative to baseline, placebo control, and/or untreated patient;

(nn) a reduction in CCL-28 levels relative to baseline, placebo control, and/or untreated patient; and

(oo) a reduction in IL-24 levels relative to baseline, placebo control, and/or untreated patient.

In one embodiment, methods of the present invention comprise measuring baseline levels of one or more markers or parameters set forth in (a)-(oo) above prior to dosing the subject or subject group. In another embodiment, the methods comprise administering a composition as disclosed herein to the subject after baseline levels of one or more markers or parameters set forth in (a)-(oo) are determined, and subsequently taking an additional measurement of said one or more markers.

In another embodiment, upon treatment with a composition of the present invention, for example, over a period of about 1 to about 12 weeks, about 1 to about 8 weeks, or about 1 to about 4 weeks, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, or all 22 of outcomes (a)-(oo) described immediately above.

In another embodiment, upon treatment with a composition of the present invention, the subject or subject group exhibits one or more of the following outcomes:

(a) a reduction in TGF-α levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(b) a reduction in TGF-β levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(c) a reduction in EGFR levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(d) a reduction in VEGF-A by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(e) a reduction in AXL levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(f) a reduction in IL-15RA levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(g) a reduction in IL-2Rβ levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(h) a reduction in IL-7 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(i) a reduction in CCL-25 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(j) a reduction in CD6 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(k) a reduction in SLAMF1 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(l) a reduction in CD244 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(m) a reduction in ALCAM levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(n) a reduction in CCL-16 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(o) a reduction in CCL-3 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(p) a reduction in IL-2Rβ levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(q) a reduction in CXCL-9 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(r) a reduction in IL-18 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(s) a reduction in IL-18R1 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(t) a reduction in IL-18BP levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(u) a reduction in CCL-4 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(v) a reduction in CCL-3 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(w) a reduction in PI3 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(x) a reduction in CDCP1 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(y) a reduction in IL-17α levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(z) a reduction in IL-17D levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(aa) a reduction in IL-17β levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(bb) a reduction in IL-12β levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(cc) a reduction in IL-17Rα levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(dd) a reduction in IL-20 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(ee) a reduction in CXCL1 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(ff) a reduction in IL-12 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(gg) a reduction in IL-5Rα levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(hh) a reduction in IL-10 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(ii) a reduction in IL-10Rβ levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(jj) a reduction in IL-13 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(kk) a reduction in CCL-11 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(ll) a reduction in CCL-23 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(mm) a reduction in CCL-24 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient;

(nn) a reduction in CCL-28 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient; and

(oo) a reduction in IL-24 levels by at least about 0.05 fold, at least about 0.10 fold, at least about 0.15 fold, at least about 0.20 fold, at least about 0.25 fold, at least about 0.30 fold, at least about 0.35 fold, at least about 0.40 fold, at least about 0.45 fold, at least about 0.50 fold, at least about 0.55 fold, at least about 0.60 fold, at least about 0.65 fold, at least about 0.70 fold, at least about 0.75 fold, at least about 0.80 fold, at least about 0.85 fold, at least about 0.90 fold, at least about 0.95 fold, or at least about 1 fold, where the fold reduction is relative to baseline, placebo control, and/or untreated patient.

In one embodiment, a DGLA-containing composition of the invention comprises the following fatty acid fingerprint as shown in Table 1.

TABLE 1

| | |
|---|---|
| C18:1n-9 | <LOD-<5% |
| C18:2n-6 | <LOD-<5% |
| 20:3ω6 - DGLA | NLT 95 |
| Isomer A | <LOD-<5% |
| C20:4n-6 + isomerB | <LOD-<5% |
| Total unidentified related substances | NMT 2 |

In one embodiment, a DGLA-containing composition of the invention comprises the following fatty acid fingerprint as shown in Table 2.

TABLE 2

| | |
|---|---|
| Fatty Acid Profile (Area % FAMEs by GC) | |
| 20:3ω6 - DGLA | NLT 95 |
| Related Substances | |
| 20:2ω6 | <LOD-<5% |
| 20:3ω3 | <LOD-<5% |
| 20:4ω6 | <LOD-<5% |
| 20:4ω3 | <LOD-<5% |
| 20:5ω3 | <LOD-<5% |
| Total unidentified related substances | NMT 2 |

An illustrative DGLA-containing composition of the invention comprises the following fatty acid fingerprint as shown in Table 3.

TABLE 3

| | |
|---|---|
| C16:0 | <LOD-<5% |
| C18:1n-7 | <LOD-<5% |
| C18:1n-9 | <LOD-<5% |
| C18:2n-6 | <LOD-<5% |
| C18:3n-6 | <LOD-<5% |
| C20:3n-3 | <LOD-<5% |
| 20:3n-6 - DGLA | NLT 95 |
| C20:4n-6 | <LOD-<5% |
| Total unidentified related substances | NMT 2 |

In one embodiment, a DGLA-containing composition of the invention comprises the following fatty acid fingerprint as shown in Table 4.

TABLE 4

| | |
|---|---|
| Fatty Acid Profile (Area % FAMEs by GC) | |
| 20:3ω6 - DGLA | NLT 95 |
| Related Substances | |
| 16:0 | <LOD-<5% |
| 18:3n-6 alcohol methyl ether | <LOD-<5% |
| 18:3n-6 alcohol formate | <LOD-<5% |
| 16:3n-3 | <LOD-<5% |
| 18:1n-9 | <LOD-<5% |
| 18:1n-7 | <LOD-<5% |
| 19:3 | <LOD-<5% |
| 20:1n-9 | <LOD-<5% |
| 20:2n-6 | <LOD-<5% |
| 20:2n-3 + DGLA isomer | <LOD-<5% |
| 20:3n-3 | <LOD-<5% |
| 20:4n-3 | <LOD-<5% |
| Methyl 7,11,14-eicosatrienoate (DGLA isomer) | <LOD-<5% |
| 22:5n-3 | <LOD-<5% |
| Total unidentified related substances | NMT 2 |

In one embodiment, a DGLA-containing composition of the invention comprises the following fatty acid fingerprint as shown in Table 5.

TABLE 5

| | | |
|---|---|---|
| C20:3 n-6 (DGLA - triglycerides) | Min. | 30% |
| C16:0 | Max. | 26% |
| C18:0 | Max. | 12% |
| C18:1 n-9 | Max. | 15% |
| C18:2 n-6 | Max. | 15% |
| C18:3 n-6 | Max. | 5% |
| C20:4 n-6 | Max. | 1% |
| C22:0 | Max. | 5% |
| C24:0 | Max. | 15% |

### EXAMPLES

The following examples are provided to further illustrate embodiments of the present technology and are not to be interpreted as limiting the scope of the present technology. To the extent that certain embodiments or features thereof are mentioned, they are merely for purposes of illustration and, unless otherwise specified, are not intended to limit the present technology. One skilled in the art may develop equivalent means without the exercise of inventive capacity and without departing from the scope of the present technology. It will be understood that many variations can be made in the procedures herein described while still remaining within the bounds of the present technology. Such variations are intended to be included within the scope of the presently disclosed technology. As such, embodiments of the presently disclosed technology are described in the following representative examples.

### Example 1

Three batches of pharmaceutical compositions comprising DGLA and 2000 pm dl-alpha tocopherol were filled into gelatin capsules. (see Table 6).

TABLE 6

| Batch Number | DGLA (mg/Capsule) | Gelatin Description |
|---|---|---|
| E09726/1 | 250 | Standard acid bovine gelatin |
| E09726/2 | 250 | Lime bone gelatin with a lower |
| | | molecular weight (Mw) |
| E09727 | 500 | Standard acid bovine gelatin |

The capsule shells included the following excipients: gelatin, purified water, glycerol, titanium dioxide, and the processing aids lecithin and medium chain triglyceride.

Additional batches of capsules were also prepared including DGLA free fatty acid (FFA) stabilized with a nominal 2000 ppm dl-alpha tocopherol in capsules containing gelatin, polysorb or mixture of gylycerol/polysorb, purified water, titanium dioxide, and the processing aids lecithin and medium-chain triglyceride as shown in Table 7.

TABLE 7

| Batch | DGLA | | |
|---|---|---|---|
| Number | (mg/Capsule) | Gelatin Description | Plasticizer |
| E09778 | 500 | Lime bone gelatin with | Glycerol |
| | | a lower molecular | |
| | | weight (Mw) | |
| E09777/01 | 500 | Lime bone gelatin with | Polysorb (D-sorbitol |
| | | a lower molecular | and 1,4-sorbitan |
| | | weight (Mw) | sugar alcohols in |
| | | | water solution) |
| E09777/02 | 500 | Lime bone gelatin with | Gycerol + Polysorb |
| | | a lower molecular | |
| | | weight (Mw) | |
| E09777/03 | 500 | Lime bone gelatin with | Polysorb |
| | | a lower molecular | |
| | | weight (Mw) - Even | |
| | | Lower Mw (Advanced | |
| | | RXL Gelatine) | |

Capsule shell compositions for each of the batches are shown below in Tables 8 and 9.

TABLE 8

| | Unit Quantity | | | |
|---|---|---|---|---|
| | mg/capsule | | | |
| Active Substance | 500 mg | | | |
| DGLA | E09778 | E09777/01 | E09777/02 | E09777/03 |
| Wet Gelatin Shell Mass | | | | |
| Gelatin | 128.97 | 128.97 | 128.97 | 128.97 |
| | (RXL) | (RXL) | (RXL) | (RXL |
| | | | | Adv.)¹ |
| Total Glycerol | 67.70 | 2.94 | 35.32 | 2.94 |
| Polysorb | N/A | 64.76 | 32.38 | 64.76 |
| Purified Water | 100.38 | 100.38 | 100.38 | 100.38 |
| Titanium Dioxide | 2.94 | 2.94 | 2.94 | 2.94 |
| Lecithin | Trace | Trace | Trace | Trace |
| Triglycerides Medium | Trace | Trace | Trace | Trace |
| Chain | | | | |

¹RXL gelatin contains a lower number of high molecular weight polymers (~5% > 200,000 Da)

TABLE 9

| | Unit Quantity | | | |
|---|---|---|---|---|
| E09726/01 | mg/capsule | % w/w | mg/capsule | % w/w |
| Active Substance | | | | |
| DGLA | 500 | 100 | 250 | 100 |
| Wet Gelatin Shell | | | | |
| Mass² | | | | |
| Gelatin (not RXL) | 132.35 | 44.12 | 87.79 | 44.12 |
| Total Glycerol | 76.76 | 25.59 | 50.92 | 25.59 |
| Purified Water | 87.95 | 29.31 | 58.33 | 29.31 |
| Glycerol and | — | — | — | — |
| Polysorb? | | | | |
| Titanium Dioxide | 2.94 | 0.98 | 1.95 | 0.98 |
| Lecithin | Trace | Trace | Trace | Trace |
| Triglycerides | Trace | Trace | Trace | Trace |
| Medium Chain | | | | |

Stability testing of the above capsules was performed. Capsules from each batch were maintained for up to 6 months and assessed using a qualitative or quantitative USP 2040 Disintegration and Dissolution test protocol. Results are shown in Tables 10-12.

TABLE 10
Stability Data for DGLA Softgel capsules: Qualitative Rupture Test Results

| | Specification: read and record (min) | | | | | | | | | | | | | | |
|---|---|---|---|---|---|---|---|---|---|---|---|---|---|---|---|
| | Results (Months): | | | | | | | | | | | | | | |
| | | 1 | 2 | 3 | | | | | | 6 | | | | | |
| | | 40° | 40° | 25° C./ | | 30° C./ | | 40° C./ | | 25° C./ | | 30° C./ | | 40° C./ | |
| | | C./ | C./ | 60% RH | | 65% RH | | 75% RH | | 60% RH | | 65% RH | | 75% RH | |
| | 0 | 75% | 75% | Without | With | Without | With | Without | With | Without | With | Without | With | Without | With |
| Batches | N/A | RH | RH | pepsin | pepsin | pepsin | pepsin | pepsin | pepsin | pepsin | pepsin | pepsin | pepsin | pepsin | pepsin |
| E09726/01 | 7 | 6 | >30/ | 8 | N/A | 1 caps | NP | >30 | 14 | >30 | | >30 | | >30 | 2 caps |
| | | | 22 ⁽¹⁾ | | | on 6 > | | | | | | | | | on 6 > |
| | | | | | | 30 | | | | | | | | | 30 |
| E09726/02 | 4 | 4 | 4 | 3 | N/A | | | 1 caps | 6 | 1 caps | 10 | 2 caps | 9 | 12 | 15 |
| | | | | | | | | on 6 > | | on 6 > | | on 6 > | | | |
| | | | | | | | | 30 | | 30 | | 30 | | | |
| E09727 | | | >30 | 4 caps | 10 | >30 | NP | >30 | 15 | >30 | 14 | >30 | 16 | >30 | 3 caps |
| | | | | on 6 > | | | | | | | | | | | on 6 > |
| | | | | 30 | | | | | | | | | | | 30 |

TABLE 11
DGLA glyceride percentages

| Batch | Time | 25 | 30 | 40 |
|---|---|---|---|---|
| Time point (months) | | DGLA glycerides % | | |
| E09727 | 0 | 0 | 0 | 0 |
| | 1 | 0 | 0 | 0.54 |
| | 2 | 0 | 0 | 1.19 |
| | 3 | 0.41 | 0.67 | 2.1 |
| | 6 | 0.53 | 1.43 | 5.81 |
| E09726/1 | 0 | 0 | 0 | 0 |
| | 1 | 0 | 0 | 0.56 |
| | 2 | 0 | 0 | 1.35 |
| | 3 | 0.37 | 0.58 | 2.31 |
| | 6 | 0.68 | 1.42 | 6.8 |
| E09726/2 | 0 | 0 | ND | 0 |
| | 1 | 0 | ND | ND |
| | 2 | 0 | ND | 1.63 |
| | 3 | 0.51 | ND | ND |
| | 6 | 1.16 | ND | 8.55 |

TABLE 12

| | | | | | Mono, Di and | | | |
|---|---|---|---|---|---|---|---|---|
| | | | | | Triglycerides | DGLA | Quantitative | |
| | | Storage | | Acid | Assay (% DGLA) | Esters (%) | Rupture test | |
| | | Conditions | | Value | Read and | Read and | Read and record | |
| Specifications | | 40° C./75% RH | | 176-184 | record | record | (mean %) | |
| | | | | | | | | Tier I |
| E09778 | (RXL + | 1 | month | 178 | 0.98 | 1.8 to 2.6² | 15 min | 94 |
| | Glycerol) | | | | | | 30 min | 102 |
| | | | | | | | 45 min | 102 |
| | | | | | | | 60 min | 102 |
| | | | | | | | | Tier II |
| | | | | | | | | stage 2 |
| | | 3 | months | 172 | 3.08 | N/A | 15 min | N/A |
| | | | | | | | 30 min | 92 |
| | | | | | | | 45 min | 96 |
| | | | | | | | 60 min | 97 |
| | | | | | | | | Tier I |
| E09777/01 | (RXL + | 1 | month | 180 | N/A | 1.8 to 2.9² | 15 min | 81 |
| | Polysorb) | | | | | | 30 min | 91 |
| | | | | | | | 45 min | 96 |
| | | | | | | | 60 min | 102 |
| | | | | | | | | Tier II |
| | | 3 | months | 182 | N/A | Not available | 15 min | N/A |
| | | | | | | | 30 min | 97 |
| | | | | | | | 45 min | 99 |
| | | | | | | | 60 min | 98 |
| | | | | | | | | Tier I |
| E09777/02 | (RXL + | 1 | month | 177 | <0.5 | 0.0 to 3.4² | 15 min | 95 |
| | Glycerol + | | | | | | 30 min | 101 |
| | Polysorb) | | | | | | 45 min | 101 |
| | | | | | | | 60 min | 102 |
| | | | | | | | | Tier I |
| | | 3 | months | 177 | 3.1 | Not avalable | 15 min | 92 |
| | | | | | | | 30 min | 97 |
| | | | | | | | 45 min | 97 |
| | | | | | | | 60 min | 98 |
| | | | | | | | | Tier I |
| E09777/03 | (RXL adv. + | 1 | month | 171⁽²⁾ | N/A | 2.7 to 3.6² | 15 min | 88 |
| | Polysorb) | | | | | | 30 min | 97 |
| | | | | | | | 45 min | 99 |
| | | | | | | | 60 min | 99 |
| | | | | | | | | Tier I |
| | | 3 | months | 174 | N/A | Not Available | 15 min | 87 |
| | | | | | | | 30 min | 94 |
| | | | | | | | 45 min | 95 |
| | | | | | | | 60 min | 95 |

As seen above, there was a reduction in dissolution rate in water over time for capsules formulated with glycerol and standard acid bovine gelatin (E09726/01, and E09727). There was a DGLA release rate of greater than 30 mins after 6 months at 40° C./75% RH in simulated gastric fluid (pH 1.2, pepsin).

A DGLA release rate of less than 30 mins after 6 months at 40° C./75% RH was only achieved in simulated gastric fluid (pH 1.2, pepsin) with capsules containing lime bone gelatin with a lower molecular weight (Mw) (E09777/02).

There was a significant increase in DGLA glyceride formation over time in DGLA capsule shells containing glycerol (Table 9). This was temperature dependent with highest concentrations of DGLA formed at 40° C. 75% RH.

Polysorb is commonly used as a hydrophilic plasticizer to limit exchange between capsule fill media and shell. D-Sorbitol and 1,4-sorbitan have a higher MW than glycerol which limits its mobility through the gelatin shell. Despite this, there was still interaction of D-Sorbitol and 1,4-sorbitan to form DGLA FFA esters in batches E09777 1/2 and 3.

There was no reduction in acid value of the DGLA for batches formulated with D-Sorbitol and 1,4-sorbitan (E097771/02/3) whereas there was a reduction in acid value for E09778 formulated with glycerol.

There was no slowdown in dissolution rate in water over time for capsules formulated with D-Sorbitol and 1,4-sorbitan (E09777/03). The DGLA release rate was less than 30 minutes in water after 3 months 40° C./75% RH.

### Example 2

A randomized, placebo-controlled, double-blind, parallel group, multi-center 3-arm Phase 2b study was performed to investigate the efficacy of orally administered a capsule containing DGLA (DS107) and the dose-response relationship between DS107 capsules and placebo in atopic dermatitis (AD) patients 18 years and older.

Investigational Medicinal Product (IMP): DS107 capsules and placebo capsules will be used in this study. Each DS107 capsule contains 500 mg DGLA as an active ingredient in an opaque, oval soft gelatin capsule. Each matching placebo capsule contains 500 mg of liquid paraffin in an opaque oval soft gelatin capsule.

DS107 capsules will be supplied in manufactured form (blinded), packaged in cold formed aluminum foil blisters of 28 units. Placebo will be presented in identical blisters and packs and stored/packaged the same as DS107 capsules. Study medication will be labelled according to US regulations.

The study objectives included:
Efficacy Objective: The efficacy objective was to compare the efficacy of orally administered DS107 versus placebo, in the treatment of adult patients with moderate to severe AD.
Safety Objective: The safety objective was to assess the safety of orally administered DS107 versus placebo, in adult patients with moderate to severe AD.

The study endpoints included:
Primary Objective: The primary object of this study was for a proportion of patients achieving an Investigator's Global Assessment (IGA) of 0 (clear) or 1 (almost clear) and a decrease of at least 2 points in IGA in treated population compared to placebo population at Week 8.
Secondary Objective: The secondary objectives of this study were the following:
   Proportion of patients achieving an IGA score of 0 (clear) or 1 (almost clear) and a decrease of at least 2 points in IGA in treated population compared to placebo population from baseline to Week 2, 4, 6 and 10;
   Proportion of patients achieving a decrease of at least 2 points in IGA in treated population compared to placebo population from baseline to Week 2, 4, 6, 8 and 10;
   Change from baseline in Eczema Area and Severity Index (EASI) in treated population compared to placebo population at Week 2, 4, 6, 8 and 10; and
   Change from baseline in Numeric Rating Scale (NRS) for Pruritus in treated population compared to placebo population at Week 2, 4, 6, 8 and 10.
Exploratory Endpoints: The exploratory endpoints of this study were the following:
   Change from baseline in the Dermatology Life Quality Index (DLQI) score in treated population compared to placebo population at Week 2, 4, 6, 8 and 10.
   Change from baseline in the Patient Orientated Eczema Measure (POEM) score in treated population compared to placebo population at Week 2, 4, 6, 8 and 10.
   change from baseline in the Patient Global Impression of Severity (PGI-S) score in treated population compared to placebo population at Week 2, 4, 6, 8 and 10.
   Change from baseline in the Patient Global Impression of Change (PGI-C) score in treated population compared to placebo population at Week 2, 4, 6, 8 and 10.
   Plasma DGLA concentrations in treated population compared to placebo population at Baseline/Day 0, Week 4, Week 8 and Week 10 of (samples to be retained and analyzed for the potential analysis at a later date).
   Determination of AD biomarkers at Baseline/Day 0, and Week 8 (samples to be retained and analyzed at a later date for the potential analysis).

Study Design: In general, all patients signed an informed consent and underwent screening for study eligibility. Patients were randomized (1:1:1) at baseline visit to either receive 2 g DS107, 1 g DS107 or placebo once daily for 8 weeks.

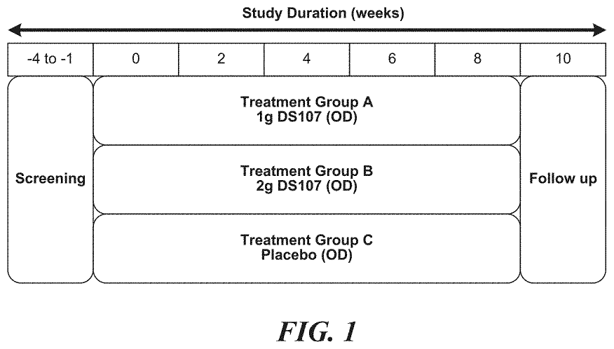

During the study, the patients came to the clinic on 7 occasions: at Screening/Visit 1, Baseline/Visit 2, Week 2/Visit 3, Week 4/Visit 4, Week 6/Visit 5 and /Week 8/Visit 6 (end of treatment) and Week 10/Visit 7 (follow-up). Early termination visits were recorded for patients who withdrew from the study early. All patients exited (i.e., finished) the study at the Week 10 visit. At the screening visit, after giving informed consent to participate, patients were assessed using the screening examinations. Patients who meet the inclusion criteria and who do not meet the exclusion criteria were enrolled. A schematic diagram of the overall timeframe of the study is provided in FIG. 1 where OD refers to once per day.

Before the comparative treatment period commenced, patients returned to the site for a baseline assessment of their disease and eligible patients were randomly allocated to one of the three parallel group treatment regimens in a 1:1:1 randomization:
Treatment group A: 1 g DS107 (2 DS107 capsules and 2 placebo capsules) administered once-daily for 8 weeks.
Treatment group B: 2 g DS107 (4 DS107 capsules) administered once-daily for 8 weeks.
Treatment group C: Placebo (4 placebo capsules) orally administered once-daily for 8 weeks.

To maintain the double-blind conditions, DS107 capsules and Placebo capsules were identical in appearance.

Unlike other oral treatments for AD (e.g. steroids) a treatment effect of DS107 capsules is not immediately expected to occur after start of treatment, but rather, to increase constantly over time reaching its maximum effect within a time period over 4-6 weeks. Therefore, every effort was undertaken to continue treatment with DS107 at least for 4 weeks to determine the efficacy of DS107. Patients who were not willing to continue to participate in the study due to the apparent lack of efficacy within the first 4 weeks of treatment therefore were replaced.

Once patients were enrolled in the study they were restricted from using any other treatment for AD, with the exception of emollients. Any medication (i.e., prescription as well as over the counter (OTC) drugs or therapeutic intervention deemed necessary for the patient, and which in the opinion of the Investigator do not interfere with the safety and efficacy evaluations, the patient was allowed to continue unless they were included in the list of ‘Concomitant Medications’ detailed below.

Patients and Screenings: Participants in this study were required to have met the following inclusion criteria and must have not met any of the following exclusion criteria. In addition, patients were only permitted to screen for this study if they were free of any sub-clinical infection, which was indicated by the presence of an eosinophil count below 0.3×109/L within 1 month of screening. The inclusion and exclusion criteria were verified at the screening visit (Visit 1) and at the start of treatment/baseline visit (Visit 2).

Source of patients: The study population consisted of male and female patients with confirmed diagnosis of AD aged 18 years or older.

Inclusion Criteria: All subjects considered for study participation were required to have met the following inclusion criteria:
Patients with a clinically confirmed diagnosis of active AD according to Hanifin and Rajka criteria discussed below.
Patients with moderate to severe AD at baseline as defined by an Investigator's Global Assessment (IGA) of minimum 3 at baseline.
Patients with an eczema Area and severity index (EASI) score of at screening and baseline.
Patients with AD covering a minimum 10% of the body surface area at baseline.
Patients whose pre-study clinical laboratory findings did not interfere with their participation in the study, in the opinion of the investigator.
Patients who were able and willing to stop all current treatments for AD throughout the study (except for allowed emollients).
Patients who were on a stable dose of a bland emollient applied BD (twice daily) for at least 7 days prior to baseline.
Male or female patients who were aged 18 years and older on the day of signing the informed consent form (ICF).
Female patients and male patients with female partners of child bearing potential must use adequate contraception or have a sterilized partner for the duration of the study. Where adequate contraception was defined as: systemic hormonal contraceptives, intrauterine device or barrier method of contraception in conjunction with spermicide, or agree to sexual abstinence. If the patient was using hormonal contraceptives, then they must have been on a stable dose for at least one month before baseline.

Exclusion Criteria: The exclusion criteria for the study were as follows:
Patients with other skin conditions that could have interfered with AD diagnosis and/or evaluation such as psoriasis or current active viral, bacterial and fungal skin infections. These conditions were assessed by the investigator.
Patients who had used systemic treatments (other than biologics) that could have affected AD less than 4 weeks prior to baseline visit (i.e., Day 0). These systematic treatments could have included for example, retinoids, methotrexate, cyclosporine, hydroxycarbamide (hydroxyurea), azathioprine and oral/injectable corticosteroids. However, Intranasal corticosteroids and inhaled corticosteroids for stable medical conditions were allowed.
Patients who had used any topical medicated treatment for AD two weeks prior to start of treatment/Baseline (Day 0), including but not limited to, topical corticosteroids, tars and bleach.
Patients who used topical products containing urea, ceramides or hyaluronic acid two weeks prior to Baseline.
Patients who used anti-histamines for AD within 2 weeks of baseline. Non-sedative anti-histamines for other indications could have been used throughout the study provided the patient was on a stable dose for 4 weeks prior to Baseline.
Patients with the presence of an active or chronic allergic reaction as evidenced by an irregular white cell count determined by eosinophils >0.3×10⁹/L at the screening visit.
Patients who have had excessive sun exposure, have used tanning booths or other ultraviolet (UV) light sources four weeks prior to Baseline and/or were planning a trip to a sunny climate or to use tanning booths or other UV sources between screening and follow-up visits.
Patients who had a history of hypersensitivity to any substance in DS107 capsules or placebo capsules.
Patients who had any clinically significant controlled or uncontrolled medical condition or laboratory abnormality that would, in the opinion of the investigator, put the patient at undue risk or interfere with interpretation of the study results.
Patients who had a clinically significant impairment of renal or hepatic function.
Patients with significant uncontrolled cardiovascular, neurologic, malignant, psychiatric, respiratory or hypertensive disease, as well as uncontrolled diabetes and fluoride arthritis or any other illness that, in the opinion of the investigator, was likely to interfere with the completion of the study.
Patients with chronic infectious disease. Examples of a chronic infectious disease are hepatitis B, hepatitis C or infection with human immunodeficiency virus.
Patients with a history of clinically significant drug or alcohol abuse in the opinion of the investigator in the last year prior to Baseline.
Patients who had participated in any other clinical study with an investigational drug within 3 months before the first day of administration of study treatment.
Patients who had treatment with biologics according to the following:
   (a) Any treatment with cell-depleting agents including but not limited to rituximab within 6 months before the screening visit, or until lymphocyte count returned to normal, whichever was longer,
   (b) Any treatment with other biologics that influence cell proliferation within 6 months before the screening visit.
Patients who are pregnant, planning pregnancy, breastfeeding and/or are unwilling to use adequate contraception during the trial.
Patients, in the opinion of the investigator who were not suitable to participate in the study.

Study Schedule: During the study, six visits to the clinic were scheduled at least one week after the screening visit: one visit was scheduled at the start of the comparative treatment period/baseline (Day 0/Visit 2) and five visits were scheduled in the comparative treatment period. These five are denoted as: Week 2/Visit 3, Week 4/Visit 4, Week 6/Visit 5, and Week 8/Visit 6. In addition, a final safety follow-up visit (denoted as Visit 7) was conducted two weeks after Week 8/Visit 6 or two weeks after the final visit attended if the patient decided not to complete the study. The baseline visit was performed, at the latest 30 days after the screening visit. Patients who discontinued the study early were asked to attend the investigative site as soon as possible so that assessments scheduled for Week 8/Visit 6 could conducted at an Early Termination visit.

In the event that the patient was receiving treatment for AD prior to the study, a wash out period of up to 4 weeks could have been necessary. At the Screening visit and after having given informed consent to participate, the patients were assessed using screening examinations. Patients were confirmed as having AD using the Hanifin and Raika criteria as described below and who did not meet the exclusion criteria at the baseline visit were enrolled in the study.

During the treatment period and follow-up period patients were restricted from using any other treatment for AD, with the exception of the same emollients the patient had been using consistently at the baseline visit. Any medication such as prescription as well as over the counter drugs or therapeutic intervention deemed necessary for the patient, and which in the opinion of the Investigator do not interfere with the safety and efficacy evaluations of the patient, the patient was allowed to continue. A list of ‘medications and therapeutic regimens excluded from the study’ is defined below.

The clinical visits for this study are provided below in Table 13.

TABLE 13
Clinical Visits Study Flow Chart

| | | | | | | Week 8/ | Week 10/ |
|---|---|---|---|---|---|---|---|
| | Screening/ | Baseline/ | Week 2/ | Week 4/ | Week 6/ | Visit 6/ Early | Visit 7/ |
| Visit | Visit 1 | Visit 2 | Visit 3 | Visit 4 | Visit 5 | Termination | Follow up |
| Day | −30 to −1 | 0 | 14 | 28 | 42 | 56 | 70 |
| Visit Window | | | +/−2 days | +/−2 days | +/−2 days | +/−2 days | +/−3 days |
| Informed Consent | X | | | | | | |
| Assign Patient identifier number | X | | | | | | |
| Demographics | X | | | | | | |
| Medical/Surgical History | X | | | | | | |
| Review Inclusion/Exclusion | X | X | | | | | |
| Criteria | | | | | | | |
| Hanifin and Rajka criteria | X | X | | | | | |
| Randomization | | X | | | | | |
| Safety labs: Serum Biochemistry | X | X | | | | X | X ² |
| (including FSH levels at screening | | | | | | | |
| when applicable¹), Hematology, | | | | | | | |
| Urinalysis | | | | | | | |
| Pharmacokinetic Sampling | | X | | X | | X | X |
| Biomarker Sampling | | X | | | | X | |
| Pregnancy Test (β-hCG if female | X | | | | | X | |
| of childbearing potential) | | | | | | | |
| Vital Signs | X | X | X | X | X | X | X |
| Physical Examination ₃ | X | X | X | X | X | X | X |
| BMI | X | X | | | | X | |
| Dispense Study Drug ₄ | | X | X | X | X | | |
| Collect Study Drug | | | X | X | X | X | |
| Dispense Patient Compliance Log | | X | X | X | X | | |
| Collect and Review Patient | | | X | X | X | X | |
| Compliance Log | | | | | | | |
| IMP Accountability | | | X | X | X | X | |
| BSA | X | X | X | X | X | X | X |
| IGA | X | X | X | X | X | X | X |
| EASI assessment | X | X | X | X | X | X | X |
| NRS Pruritus assessment ⁵ | X | X ------------------------------------------------------------------------------------------- X | | | | | |
| DLQI questionnaire | | X | X | X | X | X | X |
| POEM questionnaire | | X | X | X | X | X | X |
| PGI-S questionnaire | | X | X | X | X | X | X |
| PGI-C questionnaire | | X | X | X | X | X | X |
| Concomitant Medications | X | X | X | X | X | X | X |
| Emollient use capture ⁵ | | X ------------------------------------------------------------------------------------------- X | | | | | |
| Adverse Events ⁶ | | X | X | X | X | X | X |

¹FSH requirement to confirm female non-child-bearing potential for women greater than 40 years of age who have had a cessation of menses for at least 12 months. Non-child bearing potential may also be confirmed via cessation of menses for at least 24 months without FSH levels confirmed.
²Only if a laboratory AE recorded at Week 8. In that case only the sample which is the cause of the AE should be re-tested.
₃ Physical Examination will be symptom-directed as per section 9.2.2.
₄ Patients will be instructed to take their last study drug dose the day preceding Week 8 visit.
⁵ NRS/Emollient use will be captured electronically through the IWRS by the patient. Patients may use paper to document these assessments also.
⁶ Collection of AE will start after the first study drug administration.

Screening Visit (Visit 1): During the Screening Visit, once the patients and provided informed consist, the following screening procedures/assessments were conducted and/or obtained:
Demographic data;
Medical/Surgical history;
Assessment of inclusion/exclusion criteria;
Hanifin and Rajka criteria review;
Samples for clinical laboratory safety tests such as an assessment of hematology, serum biochemistry, urinalysis and follicle stimulating hormone (FSH) levels (when applicable);
Sample for pregnancy test (only if a female patient of child-bearing potential);
Assessment of vital signs such blood pressure, heart rate and body temperature;
Physical examination;
Body Mass Index;
Body Surface Area;
Investigator's Global Assessment;
Eczema Area and Severity Index (EASI);
NRS Pruritus Assessment; and
Concomitant medication assessment.

Unscheduled visits occurred when a patient needed to make a visit in between the scheduled visit dates due to an adverse event (AE), had a difficulty complying with the study protocol requirements, or had a significant change in their disease state.

Treatment Period: Following completion of a successful screening visit, patients began the comparative treatment period for a total of 8 weeks. At the start of the comparative treatment period and after confirmation of continued eligibility, patients were randomly assigned at the baseline visit (Visit 2) to one of the three treatment regimens (i.e., 1 g of DS107, 2 g of DS107, or a Placebo).

At the start of the treatment period, patients were instructed to take 4 capsules of investigational medicinal product (IMP) which contained either 1 g of DS107, 2 g or DS107 or a Placebo, consistent with the treatment regimens. Every effort was to ensure that IMP administration occurred at the same time each day.

Unscheduled visits occurred when a patient needed to make a visit in between the scheduled visit dates due to an adverse event (AE), had a difficulty complying with the study protocol requirements, or had a significant change in their disease state.

Baseline (Visit 2): At Visit 2, it was ensured that all inclusion regarding the severity of the disease remained unchanged from the screening visit. This step was performed in order to exclude those patients who reacted to emollient use since the screening visit. During Visit 2, the following screening procedures/assessments were conducted and/or obtained:
Verification of the inclusion/exclusion criteria;
Hanifin and Rajka criteria review;
Patient Randomization
Samples for clinical laboratory safety tests such as an assessment of hematology, serum biochemistry, and urinalysis;
Pharmacokinetic Sampling;
Biomarker Sampling;
Assessment of vital signs such blood pressure, heart rate and body temperature;
Physical examination;
Body Mass Index;
Dispensed Study Drug;
Dispensed Patient Compliance Log;
Body Surface Area;
Investigator's Global Assessment;
Eczema Area and Severity Index (EASI);
NRS Pruritus assessment;
Dermatology Life Quality Index (DLQI) Questionnaire;
Patient Orientated Eczema Measured;
Patient Global Impression of Severity Questionnaire;
Patient Global Impression of Change Questionnaire; and
Concomitant medication assessment.

The first administration of DS107 or Placebo was carried out after Visit 2. After which, the DS107 capsules or Placebo medication were administered once daily and IMP administered approximately 2 hours after food consumption at the same time each day. An NRS for the assessment of pruritus was captured on a daily basis from baseline to the follow up visit. Emollient use was captured on a daily basis from Visit to the follow up visit. The collection of AE's began after the first administration of IMP.

Week 2 (Visit 3): During Visit 3, patients returned to the investigational site at Week 2/Visit 3 and the following screening procedures/assessments were conducted and/or obtained:
Assessment of vital signs such blood pressure, heart rate and body temperature;
Physical examination;
Dispensed Study Drug;
Collected Study Drug;
Dispensed Patient Compliance Log;
Collected and Reviewed Patient Compliance Log;
IMP Accountability;
Body Surface Area;
Investigator's Global Assessment;
Eczema Area and Severity Index (EASI);
NRS Pruritus assessment;
Dermatology Life Quality Index (DLQI) Questionnaire;
Patient Orientated Eczema Measured;
Patient Global Impression of Severity Questionnaire;
Patient Global Impression of Change Questionnaire;
Concomitant medication assessment; and
AE assessment.

The IMP continued to be administered approximately 2 hours after food consumption at the same time each day. An NRS for the assessment of pruritus was captured on a daily basis from baseline to the follow up visit. Emollient use was captured on a daily basis from Visit to the follow up visit.

Week 4 (Visit 4): During Visit 4, the following screening procedures/assessments were conducted and/or obtained:
Pharmacokinetic Sampling;
Assessment of vital signs such blood pressure, heart rate and body temperature;
Physical examination;
Dispensed Study Drug;
Collected Study Drug;
Dispensed Patient Compliance Log;
Collected and Reviewed Patient Compliance Log;
IMP Accountability;
Body Surface Area;
Investigator's Global Assessment;
Eczema Area and Severity Index (EASI);
NRS Pruritus assessment;
Dermatology Life Quality Index (DLQI) Questionnaire;
Patient Orientated Eczema Measured;
Patient Global Impression of Severity Questionnaire;
Patient Global Impression of Change Questionnaire;
Concomitant medication assessment; and
AE assessment.

The IMP continued to be administered approximately 2 hours after food consumption at the same time each day. An NRS for the assessment of pruritus was captured on a daily basis from baseline to the follow up visit. Emollient use was captured on a daily basis from Visit to the follow up visit.

Week 6 (Visit 5): During Visit 5, the following screening procedures/assessments were conducted and/or obtained:
Assessment of vital signs such blood pressure, heart rate and body temperature;
Physical examination;
Dispensed Study Drug;
Collected Study Drug;
Dispensed Patient Compliance Log;
Collected and Reviewed Patient Compliance Log;
IMP Accountability;
Body Surface Area;
Investigator's Global Assessment;
Eczema Area and Severity Index (EASI);
NRS Pruritus assessment;
Dermatology Life Quality Index (DLQI) Questionnaire;
Patient Orientated Eczema Measure;
Patient Global Impression of Severity Questionnaire;
Patient Global Impression of Change Questionnaire;
Concomitant medication assessment; and
AE assessment

The IMP continued to be administered approximately 2 hours after food consumption at the same time each day. An NRS for the assessment of pruritus was captured on a daily basis from baseline to the follow up visit. Emollient use was captured on a daily basis from Visit to the follow up visit.

Week 8 (Visit 6)/End of Treatment or Early Termination: During Visit 6 the last dose of either DS107 or the Placebo were taken and the following screening procedures/assessments were conducted and/or obtained:
Samples for clinical laboratory safety tests such as haematology, serum biochemistry, and urinalysis;
Pharmacokinetic Sampling;
Biomarker Sampling;
Sample for pregnancy test (only if a female patient of child-bearing potential);
Assessment of vital signs such as blood pressure, heart rate, and body temperature;
Physical examination;
Body Mass Index;
Collected Study Drug;
Collected and Reviewed Patient Compliance Log
IMP Accountability;
Body Surface Area;
Investigator's Global Assessment;
Eczema Area and Severity Index (EASI)
NRS Pruritus assessment;
Dermatology Life Quality Index (DLQI) Questionnaire;
Patient Orientated Eczema Measured;
Patient Global Impression of Severity Questionnaire;
Patient Global Impression of Change Questionnaire;
Concomitant medication assessment; and
AE assessment

A numeric rating scale (NRS) for the assessment of pruritus was captured on a daily basis from baseline to the follow up visit. Emollient use was captured on a daily basis from Visit to the follow up visit. On completion of this visit, patients were advised that were required to return to the investigational site at Visit 7 to assess any AEs since this visit, and to conduct safety and efficacy assessments.

Week 10 (Visit 7)/Follow up: Two weeks after Visit 6 or from the early withdrawal visit, the following screening procedures/assessments were conducted and/or obtained:
Samples for clinical laboratory safety tests such as haematology, serum biochemistry, and urinalysis. However, these samples were only obtained if the patient exhibited a clinically significant change from baseline in safety lab results at Week 8/Visit 6;
Pharmacokinetic Sampling;
Assessment of vital signs such as blood pressure, heart rate, and body temperature;
Physical examination;
Body Surface Area;
Investigator's Global Assessment
Eczema Area and Severity Index (EASI);
NRS Pruritus assessment;
Dermatology Life Quality Index (DLQI) Questionnaire;
Patient Orientated Eczema Measure;
Patient Global Impression of Severity Questionnaire;
Patient Global Impression of Change Questionnaire;
Concomitant medication assessment; and
AE assessment

Assessments

The assessments for this study were as follows:

Efficacy assessments: The following efficacy assessments were included:

Investigator Global Assessment (IGA): The clinical severity of AD was evaluated by the investigator at each visit using the IGA scale provide below in Table 14.

TABLE 14

| IGA Severity | Morphological Description |
|---|---|
| 4 - Severe | Deep/dark red erythema; marked and extensive |
| | elevation (papules/infdtration). |
| 3 - Moderate | Dull, red, clearly distinguishable erythema; clearly |
| | perceptible lesion elevation (papules/infiltration) |
| | but not extensive. |
| 2 - Mild | Visibly detectable, light pink erythema and very |
| | slight elevation (papules/infiltration). |
| 1 - Almost Clear | Barely perceptible erythema and/or minimal lesion |
| | elevation (papules/infiltration). |
| 0 - Clear | No signs of erythema, lesions, papulation or |
| | infiltration |

The IGA scale awards a score of 0-4 based on a 5-point severity scale from clear to severe disease (0=clear, 1=almost clear, 2=mild disease, 3=moderate disease, 4=severe disease). IGA also uses clinical characteristics of erythema, infiltration, papulation and oozing/crusting as scoring guidelines for an overall severity assessment. The IGA was assessed at every visit.

Eczema Area Severity Index (EASI): The EASI was assessed at Screening, Baseline/Visit 2, Week 2/Visit 3, Week 4/Visit 4, Week 6/Visit 5, Week 8/Visit 6/ET and Week 10/Visit 7 (i.e, the follow-up visit). EASI quantifies the severity of a patient's AD based on both lesion severity and the percent of BSA affected. The EASI is a composite score ranging from 0-72 that takes into account the degree of erythema, induration/papulation, excoriation, and lichenification (each scored from 0 to 3 separately) for each of four body regions, with adjustment for the percent of BSA involved for each body region and for the proportion of the body region to the whole body. A detailed procedure of EASI score calculation is provided below.

Four anatomic sites—head, upper extremities, trunk and lower extremities—are assessed for erythema, induration (papules), excoriation and lichenification as seen on the day of the examination. The severity of each sign is assessed using a 4-point (half points permitted) scale:
0=none
1=mild
2=moderate
3=severe

The area affected by AD within a given anatomic site is estimated as a percentage of the total area of that anatomic site and assigned a numerical value according to the degree of AD involvement as follows:
0=no involvement
1=<10%
2=10 to 29%
3=30 to 49%
4=50 to 69%
5=70 to 89%
6=90 to 100%

The EASI score is obtained by using the formula:

EASI=0.1(Eₕ+Iₕ+Exₕ+Lₕ)Aₕ+0.2(Eᵤ+Iᵤ+Exᵤ+Lᵤ)Aᵤ+0.3(Eₜ+Iₜ+Exₜ+Lₜ)At+0.4(Eₗ+Iₗ+Exₗ+Lₗ)Aₗ

In this formula, E, I, Ex, L and A denote erythema, induration, excoriation, lichenification and area, respectively, and h, u, t, and l denote head/neck, upper extremities, trunk, and lower extremities, respectively.

Pruritus NRS: The severity of pruritus related to AD was self-assessed by patients daily using the NRS. Patients were asked to estimate the intensity of pruritus at its worst over the previous 24 hours. The Pruritus NRS is a single-question assessment tool that was used to assess the patient's worst itch as a result of AD in the previous 24 hours. Patients scored their pruritus due to AD on a scale of 0-10, with 0 (no itch) and 10 (worst itch imaginable). Patients completed the rating scale at screening and then daily starting at baseline through to the last study visit.

Body Surface Area (BSA): The overall BSA affected by AD was evaluated (from 0 to 100%) at each visit. One patient's palm represents 1% of his/her total BSA.

Dermatology Life Quality Index (DLQI) Questionnaire: The effect of AD on patient quality of life will be self-assessed by the patient at Baseline/Visit 2, Week 2/Visit 3, Week 4/Visit 4, Week 6/Visit 5, Week 8/Visit 6/ET and Follow up Week 10/Visit 7 using the DLQI. DLQI has a maximum value of thirty based on the patient's response to ten questions scored according to the following scale:
Very Much=3
A lot=2
A little=1
Not at all=0
Not relevant=0
Question unanswered=0
Question 7: “prevented work or studying”=3

DLQI is determined using the following questionnaire:

The aim of this questionnaire is to measure how much your skin problem has affected
your life OVER THE LAST WEEK. Please check one box for each question.

| | | | | |
|---|---|---|---|---|
| 1. | Over the last week, how itchy, sore, painful or stinging | Very much | □ | |
| | has your skin been? | A lot | □ | |
| | | A little | □ | |
| | | Not at all | □ | |
| 2. | Over the last week, how embarrassed or self conscious | Very much | □ | |
| | have you been because of your skin? | A lot | □ | |
| | | A little | □ | |
| | | Not at all | □ | |
| 3. | Over the last week, how much has your skin interefered | Very much | □ | Not relevant □ |
| | with you going shopping or looking after your home or | A lot | □ | |
| | yard? | A little | □ | |
| | | Not at all | □ | |
| 4. | Over the last week, how much has your skin influenced | Very much | □ | Not relevant □ |
| | the clothes you wear? | A lot | □ | |
| | | A little | □ | |
| | | Not at all | □ | |
| 5. | Over the last week, how much has your skin affected any | Very much | □ | Not relevant □ |
| | social or leisure activities? | A lot | □ | |
| | | A little | □ | |
| | | Not at all | □ | |
| 6. | Over the last week, how much has your skin made it | Very much | □ | Not relevant □ |
| | difficult for you to do any sport? | A lot | □ | |
| | | A little | □ | |
| | | Not at all | □ | |
| 7. | Over the last week, has your skin prevented you from | yes | □ | Not relevant □ |
| | working or studying? | no | □ | |
| | If “No”, over the last week how much has your skin been | A lot | □ | |
| | a problem at work or studying | A little | □ | |
| | | Not at all | □ | |
| 8. | Over the last week, how much has your skin created | Very much | □ | Not relevant □ |
| | problems with your partner or any of your close friends | A lot | □ | |
| | or relatives? | A little | □ | |
| | | Not at all | □ | |
| 9. | Over the last week, how much has your skin caused | Very much | □ | Not relevant □ |
| | any sexual difficulties? | A lot | □ | |
| | | A little | □ | |
| | | Not at all | □ | |
| 10. | Over the last week, how much of a problem has the | Very much | □ | Not relevant □ |
| | treatment for your skin been, for example by making your | A lot | □ | |
| | home messy, or by taking up time? | A little | □ | |
| | | Not at all | □ | |

©A Y Finlay, G K Khan, April 1992 www.dermatology.org.uk.
Please check you have answered EVERY question. Thank you.

Patient Orientated Eczema Measure (POEM): The POEM was assessed at each visit, except the screening visit. The POEM is a self-assessment of disease severity by the patient. POEM has a maximum value of twenty-eight based on the patient's response to seven questions scored according to the following scale:
No Days=0
1-2 Days=1
3-4 Days=2
5-6 Days=3
Everyday=4

POEM is determined using the following questionnaire:

| | |
|---|---|
| Patient ID #: - | Patient Initials: |

| | |
|---|---|
| Visit Day: | Visit Date (dd-mmm-yyyy): |

Please circle one response for each of the seven questions below about your eczema. Please
leave blank any questions you feel unable to answer.
Please circle one response for each of the seven questions below about your eczema. Please
leave blank any questions you feel unable to answer.
1. Over the last week, on how many days has you skin been itchy because of your eczema?

| | | | | |
|---|---|---|---|---|
| No days | 1-2 days | 3-4 days | 5-6 days | Every day |

2. Over the last week, on how many nights has your sleep been disturbed because of your
eczema?

| | | | | |
|---|---|---|---|---|
| No days | 1-2 days | 3-4 days | 5-6 days | Every day |

3. Over the last week, on how many days has your skin been bleeding because of your eczema?

| | | | | |
|---|---|---|---|---|
| No days | 1-2 days | 3-4 days | 5-6 days | Every day |

4. Over the last week, on how many days has your skin been weeping or oozing clear fluid
because of your eczema?

| | | | | |
|---|---|---|---|---|
| No days | 1-2 days | 3-4 days | 5-6 days | Every day |

5. Over the last week, on how many days has your skin been cracked because of your eczema?

| | | | | |
|---|---|---|---|---|
| No days | 1-2 days | 3-4 days | 5-6 days | Every day |

6. Over the last week, on how many days has your skin been flaking off because of your eczema?

| | | | | |
|---|---|---|---|---|
| No days | 1-2 days | 3-4 days | 5-6 days | Every day |

7. Over the last week, on how many days has your skin felt dry or rough because of your eczema?

| | | | | |
|---|---|---|---|---|
| No days | 1-2 days | 3-4 days | 5-6 days | Every day |

Patient Global Impression of Severity (PGI-S): The self-report PGI-S is a global index that may be used to rate the severity of a specific condition (i.e., a single-state scale). PGI-S is a simple, direct, easy to use scale that is intuitively understandable to clinicians. The PGI-S involves asking a patient a single question and rating how their AD is now on a scale of 1 (Normal Skin) to 4 (Severe). PGI-S was assessed by the patient at Baseline/Visit 2, Week 2/Visit 3, Week 4/Visit 4, Week 6/Visit 5, Week 8/Visit 6/ET and Follow up Week 10/Visit 7.

Patient Global Impression of Change (PGI-C): The self-report measure PGI-C reflects a patient's belief about the efficacy of treatment. PGI-C is a 7-point scale depicting a patient's rating of overall improvement. Patients rate their change as “very much improved,” “much improved,” “minimally improved,” “no change,” “minimally worse,” “much worse,” or “very much worse.” PGI-C was assessed by the patient at Baseline/Visit 2, Week 2/Visit 3, Week 4/Visit 4, Week 6/Visit 5, Week 8/Visit 6/ET and Follow up Week 10/Visit 7.

Safety Assessments:

A complete review of the patient's medical history was undertaken by the Investigator or designee at the Screening visit (Visit 1) to ensure that no exclusion criteria had been met. Any concomitant disease, whether considered relevant for the study or not by the Investigator, must be reported. The date of diagnosis or duration of the condition should be noted where possible.

A physical examination was performed by the investigator as per the Study Flow Chart (See Table 13) at every visit in accordance with local practices. This examination was symptom-directed, that is a standard panel of body systems were not assessed unless indicated by patient. For example, should the patient report to the investigator the presence of a ‘rash’ then the skin was evaluated. However, it is not required that additional body systems are assessed unless clinically warranted. Any clinically significant abnormal results should be recorded. Further, changes in findings of the physical examination compared with the baseline examination were recorded as an AE.

Vital signs measurements were performed as per the Study Flow Chart (See Table 13) at every visit. Measurements included:
Blood pressure: will be performed as supine (after at least 5 minutes of rest) systolic and diastolic blood pressure (in mmHg)
Heart rate: taken at rest (in bpm)
Temperature: will be taken as per clinic practice. Temperature and route will be recorded in the CRF.

The vital signs measurements were performed before any blood samples were taken. All new findings or changes to previous findings considered clinically significant were recorded as an AE.

Clinical laboratory tests included the following Haematology, Serum Biochemistry, and Urinalysis. For the clinical laboratory safety tests, blood and urine samples were taken as per the Study Flow Chart (See Table 13) for routine hematology, serum biochemistry and urinalysis tests.

TABLE 15
Clinical Laboratory Safety Tests

| | |
|---|---|
| Haematology: | Full blood count to include red cell count, |
| | haemoglobin, haematocrit, white cell count, |
| | differential white cell count, platelet count |
| | and. |
| Serum | Urea (blood urea nitrogen; BUN), creatinine, |
| biochemistry: | uric acid, total bilirubin, potassium, alkaline |
| | phosphatase (ALP), aspartate aminotransferase |
| | (AST), alanine aminotransferase (ALT), lactate |
| | dehydrogenase (LDH), creatine phosphokinase |
| | (CPK), albumin, total protein, cholesterol, |
| | triglycerides, glucose. |
| Urinalysis: | pH, protein, glucose, blood. |

If Follicule-Stimulating Hormone (FSH) test was required to confirm non-child bearing potential, this test was only carried out at screening.

For female patients of childbearing potential, a pregnancy test was carried out at screening and Week 8/Visit 6/ET visit, as per the Study Flow Chart (See Table 13).

Weight (kg) and Height (cm) were collected to calculate the BMI (kg/m²), and were recorded at Screening/Visit 1, Baseline/Visit 2 and Week 8/Visit 6/ET. The height was only recorded once at the screening visit and the same value was used for BMI calculation at Baseline/Visit 2 and Week 8/Visit 6 visits.

Blood samples for pharmacokinetic (PK) analysis were collected via direct venepuncture as per the Study Flow Chart (See Table 13) at Baseline/Visit 2, Week 4/Visit 4, Week 8/Visit 6/ET and Week 10/Visit 7/Follow up.

Biomarker Sampling: Blood samples were collected via direct venepuncture as per the Study Flow Chart (See Table 13) at Baseline/Visit 2 and Week 8/Visit 6/ET.

Any medication such as prescription as well as OTC drugs, vitamins and antacids or therapeutic intervention deemed necessary for the patient, and which, in the opinion of the Investigator, do not interfere with the safety and efficacy evaluations, could be continued by the patient unless they are included in the list of ‘medications and therapeutic regimens excluded from the study’ outlined below. Any medications, herbal medicines, natural health remedies and nutritional supplements used within 30 days prior to Screening (Visit 1) until completion (Visit 7) were recorded. The generic name of the medication (i.e., not local trade names), along with start date, stop date, dose, route, regimen and indication was also recorded. Any new medications or changes to the dose or regimen of pre-existing medications were updated on a routine basis during the study. Investigational new drugs (i.e. drugs that were not marketed in the local market) were not co-administered with the IMPs during the entire period of the study.

The permitted therapies during this study were as follows:

All patients applied a bland emollient of their choice, initiated at least 7 days prior to Baseline/Day 0, twice a day on their skin including AD lesions. Emollient use was required to continue at the same frequency and on the same skin areas throughout the study. Patients were requested to avoid using emollients containing any active ingredient which has or may have an effect on AD including the following ingredients:
Urea
Ceramide
Hyaluronic acid

Every effort was made to keep the same emollient throughout the study. The commercial name of the selected emollient(s) was recorded, along with the frequency and quantity. Patients also recorded on a daily basis their emollient use as instructed by the clinic staff. No other products may be applied to the lesions during the study.

Other Permitted Therapies: Non-sedative anti-histamines (e.g. loratadine, fexofenadine) are allowed during the study only if used to treat medical conditions other than AD. Such medications are allowed during the study only if the patient has been on a stable dose for at least 4 weeks prior to Baseline/Day 0 and continues to use the same agent everyday throughout the study. Inhaled and intranasal corticosteroids for stable medical conditions are allowed.

Medications and therapeutic regimens were excluded from use in the study:
Topical medicated treatments that could affect AD, including but not limited to:
   topical corticosteroids
   tars
   bleach, and
   any topical product containing urea, ceramides or hyaluronic acid.

Systemic therapy that could affect AD, e.g. retinoids, methotrexate, cyclosporine, hydroxycarbamide (hydroxyurea), azathioprine and oral/injectable corticosteroids; anti-histamines used for AD; any biological agent; UV-A or UV-B phototherapy; psoralen+Ultraviolet A (PUVA) therapy; use of tanning booth; any other investigational medicinal product; or traditional medicine, herbal extracts and supplements used to treat AD.

Patients were asked to refrain from any travel to sunny climates or use of tanning equipment, saunas and swimming throughout the duration of the study. Extensive UV exposure or UV-B devices will be prohibited within 4 weeks of the start of the trial and during the trial.

Patients were instructed to abstain from using any drugs/treatments that may influence AD (refer to exclusion criteria and prohibited therapies or procedures section) throughout the study. Patients will be required to administer the drug 2 hours after the consumption of food. Medication(s) for other conditions that are permitted in the study can be taken as usual.

Investigational Medicinal Product/Investigational Drug: Dosage and Administration

This study involves a comparison of DS107 (2 g and 1 g) with placebo, administered orally once daily for a total duration of 8 weeks. The last study drug administration should occur on the day preceding Week 8 (Visit 6)/Early Termination (ET) visit. Patients will be randomized to one of the three treatment groups in a 1:1:1 ratio:
Treatment group A: 1 g DS107 (2 DS107 capsules and 2 placebo capsules) administered once-daily for 8 weeks
Treatment group B: 2 g DS107 (4 DS107 capsules) administered once-daily for 8 weeks
Treatment group C: Placebo (4 placebo capsules) orally administered once-daily for 8 weeks

Patients will be required to administer the drug 2 hours after the consumption of food. Medication(s) for other conditions that are permitted in the study can be taken as usual.

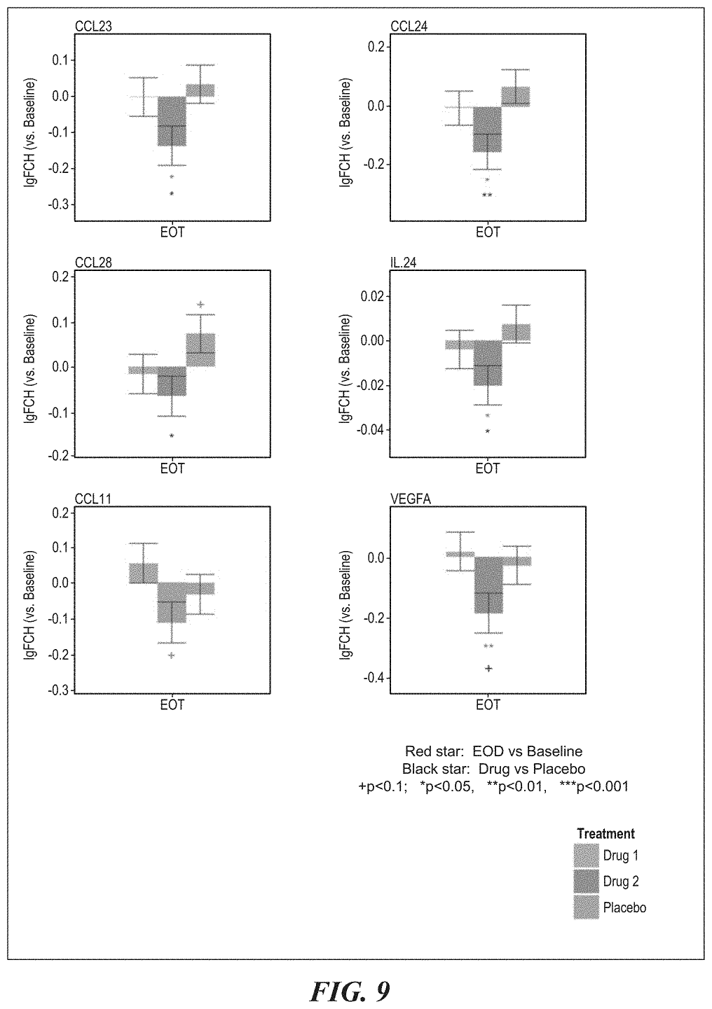

Blister packs will consist of 7 rows of 4 capsules with each weekday detailed. Each row constitutes one dose. Patients will be instructed to take the 4 capsules from left to right, on the relevant day, as shown in FIG. 9: To maintain the blind throughout the study, the DS107 capsules and placebo capsules will be identical in appearance. Patients will be orally administered DS107 or placebo once daily for 8 consecutive weeks.

Adverse Events and Serious Adverse Events

Adverse Events (AE): Any undesirable experience occurring to a patient who has taken their first dose of the study drug, whether or not considered related to the investigational IMP(s).

Serious Adverse Events (SAE): If a patient experiences a serious adverse event after the first dose of the study drug, the event will be recorded as a serious adverse event.

A serious adverse event (experience) or reaction is any untoward medical occurrence that at any dose:
results in death;
is life-threatening;
requires in-patient hospitalization or prolongation of existing hospitalization;
results in persistent or significant disability/incapacity, or
is a congenital anomaly/birth defect

The term “life-threatening” in the definition of “serious” refers to an event in which the patient was at risk of death at the time of the event. It does not refer to an event which hypothetically might have caused death if it were more severe.

Medical and scientific judgment should be exercised in deciding whether expedited reporting is appropriate in other situations, such as important medical events that may not be immediately life-threatening or result in death or hospitalization but may jeopardize the patient or may require intervention to prevent one of the other outcomes listed in the definition above. These should also usually be considered serious.

Examples of such events are intensive treatment in an emergency room or at home for allergic bronchospasm, blood dyscrasias or convulsions that do not result in hospitalization, or development of drug dependency or drug abuse.

Severity: The intensity of an AE is an estimate of the relative severity of the event made by the investigator based on his or her clinical experience. The following definitions are to be used to rate the severity of an AE:
Mild: The adverse event is transient and easily tolerated.
Moderate: The adverse event causes the patient discomfort and interrupts the patient's usual activities.
Severe: The adverse event causes considerable interference with the patient's usual activities, and may be incapacitating or life-threatening.

Relationship to IMP

The investigator will establish causality of the AE to experimental treatment. The investigator should take into account the patient's history, most recent physical examination findings, and concomitant medications.

The following definitions will be used to determine causality of an AE:
Not related: temporal relationship of the onset of the AE, relative to the experimental treatment is not reasonable or another cause can explain the occurrence of the AE.
Related: temporal relationship of the onset of the AE, relative to the experimental treatment is reasonable, follows a known response pattern to the treatment, and an alternative cause is unlikely.

Reporting AEs and SAEs: All AEs must be recorded in the case report form, defining relationship to IMP and severity. The frequency of each AE should always be recorded to indicate if the event is intermittent, continuous, one-time event etc. If the same AE occurs repeatedly at approximately the same strength in the same patient this AE should be counted only once. If any aspect of the AE changes (including but not limited to severity, frequency, causality) a new AE should be recorded.

Serious Adverse Reactions and Unexpected Adverse Reactions: Adverse Reaction: All noxious and unintended responses to a medicinal product related to any dose should be considered adverse drug reactions. The phrase “responses to a medicinal product” means that a causal relationship between a medicinal product and an adverse event is at least a reasonable possibility i.e. the relationship cannot be ruled out. For marketed medicinal products, an adverse reaction is a response to a drug which is noxious and unintended and which occurs at doses normally used in man for prophylaxis, diagnosis, or therapy of disease or for modification of physiological function.

Unexpected Adverse Reaction: An adverse reaction, the nature or severity of which is not consistent with the applicable product information

Suspected Unexpected Serious Adverse Reaction (SUSAR): Any serious adverse reaction that might be related to the IMP and are unexpected according to the definition above.

Differentiation of Treatment Failure and AE: The lack of improvement of the symptoms of AD is not an AE and should be considered a treatment failure.

Pregnancy Reporting: If a patient or a patient's partner becomes pregnant during the study, the patient should inform the study site as soon as possible. Upon confirmation of the pregnancy, the patient must be withdrawn from study drug but may continue study participation. Post-treatment follow-up should be done to ensure patient safety. Pregnancy is not itself an AE or SAE, however maternal/fetal complications or abnormalities will be recorded as AEs or SAEs as appropriate.

Estimation of Sample Size: In the Phase 2a trial, IGA response rates of 21.6% and 11.8% were observed for DS107 and Placebo respectively. Assuming these rates and a significance of 5%, 300 evaluable patients (100 patients per treatment arm) will yield 45% power for comparisons between the active doses and placebo in this study.

Interim Analysis: An interim analysis will be conducted once 50% of planned patients have completed their Week 8 assessments.

Analysis Populations
Enrolled Population: The Enrolled Population consists of all patients who sign informed consent.
Screen Failures: Screen Failures are patients from the Enrolled Population who do not meet the eligibility requirements and are withdrawn from the study prior to Randomization.
Randomized Population: The Randomized Population consists of all patients who are randomized to the study.
Safety Analysis Set (SAS): The Safety Analysis Set (SAS) consists of all patients who received at least one dose of the medication. SAF is the analysis population for all safety endpoints. Analysis will be done according to the actual treatment patients received.
Full Analysis Set (FAS): The Full Analysis Set (FAS) consists of all patients who are randomized to the study and received at least one dose of study medication. FAS is the primary analysis population for efficacy endpoints. Analysis will be done according to the treatment patients were randomized to.
Per Protocol Set (PPS): The Per Protocol Set (PPS) is the subset of FAS who completed the study without any major violations. Protocol violations will be assessed for each patient in a blinded fashion prior to database lock at a Blind Data Review Meeting (BDRM), and the PPS will also be finalized during this meeting. PPS is a supportive analysis population for efficacy endpoints. Analysis will be done according to the treatment patients were randomized to.

Safety Analysis: Demographic, medical history and physical examination data will be listed for each patient and summarized descriptively.

All AEs recorded during the study will be coded to system organ class and preferred terms using the current version of the Medical Dictionary for Regulatory Activities (MedDRA). AEs will be tabulated and summarized by treatment, relationship to treatment and severity.

Clinical laboratory values (hematology, biochemistry, and urinalysis) will be listed for each patient by treatment and day. Values outside the laboratory normal ranges will be listed separately with associated comments as to their clinical significance, with potentially clinically significant abnormalities highlighted and summarized by treatment. Clinical laboratory values obtained prior to dosing will be defined as baseline values. Individual values of vital signs will be listed and summarized descriptively for each treatment and day.

Pharmacokinetic Analysis: Plasma concentrations of DGLA will be tabulated and summarized descriptively. Individual and mean plasma concentration-time profiles of DGLA will be presented graphically.

Primary variables: The primary variable will be the proportion of patients achieving an IGA score of 0 (clear) or 1 (almost clear) and a decrease of at least 2 points in IGA from baseline at Week 8.

The primary endpoint will be analyzed using a Generalized Linear Mixed Model (GLMM) with treatment arm and baseline IGA value as factors, with the treatment-by-visit interaction term as the random effect to account for missing data. The primary analysis will be based on the FAS, and repeated for the PPS as a supportive sensitivity analysis.

The primary statistical analysis assumes “Missing At Random (MAR)” when handling missing data. The treatment effect obtained under the MAR assumption is essentially that which could have been reached if all patients had fully adhered to treatment or, in other words, the effect a patient may expect if they take the medication as directed. This is sometimes known as the ‘de jure’ or ‘efficacy’ estimand. Because of the lack of perfect adherence in practice, the ‘de facto’ or effectiveness treatment effect will also be estimated. This estimand includes assumptions regarding the treatment effects that could be expected to occur when patients discontinue treatment. The jump to reference method described by Carpenter et al. (J Biopharm Stat, 2013; 23(6):1352-71) will be used to estimate the de facto estimand, using the Placebo arm as the reference. This is based on the assumption that patients who discontinue from study drug have no alternative oral treatment option suitable for longer-term use and so their responses are likely to revert to those of the Placebo group. The sensitivity analyses for missing data will be performed on the FAS only.

Secondary variables: IGA-responders at other time points will also be analyzed using a GLMM model. The continuous efficacy variables and their changes from baseline will be summarized with descriptive statistics per treatment group and visit. This applies to the IGA, EASI, NRS scores for pruritus, DLQI, POEM, PGI-S and PGI-C. Change from baseline endpoints will be analyzed using Mixed Model with Repeated Measures (MMRM) with Treatment Arm as a factor and baseline value as a covariate, and with the treatment-by-visit interaction term as the repeated effect to account for missing data. The secondary efficacy analyses will be based on the FAS only.

Safety variables: The type and frequency of adverse events will be summarized by MedDRA system organ class and preferred term per treatment group. In addition, the number and proportion of patients with at least one adverse event will be summarized per treatment group. The number and proportion of patients experiencing serious adverse events, adverse events leading to withdrawal and adverse events possibly or probably related to treatment will be summarized per treatment group. The secondary safety analysis will be based on the Safety Analysis Set only.

Monitoring/Quality Control: Monitoring visits will be conducted during the study at regular intervals. The monitoring visits will be conducted to ensure protocol adherence, quality of data, accuracy of entries in the eCRF, drug accountability, compliance with regulatory requirements and continued adequacy of the investigational site and its facilities. All clinical data will undergo quality control checks prior to clinical database lock. Edit checks will then be performed for appropriate databases as a validation routine using SAS® to check for missing data, data inconsistencies, data ranges etc.

End of Trial: End of Trial is defined as Last Subject Last Visit (LSLV). LSLV is defined as the date the investigator reviews the last subject's safety data and determines that no further evaluation is required for the subject to complete the trial.

Diagnostic Features of Atopic Dermatitis (Hanifin and Rajka Criteria): Patients must have confirmed diagnosis of AD based on the Hanifin and Rajka diagnostic criteria. Firm diagnosis of AD requires the presence of at least three of the major criteria described below. In addition to having three of the major criteria, a patient should manifest three minor criteria which are either less specific or relatively rare.

Major criteria:
Pruritus
Dermatitis affecting flexural surfaces in adults and the face and extensors in infants and children
Chronic or relapsing dermatitis
Personal or family history of cutaneous or respiratory atopy (asthma, allergic rhinitis, AD).

Minor criteria can be divided into 4 categories:
Facial features: facial pallor, facial erythema, hypopigmented patches, infraorbital darkening, infraorbital folds (Dennie-Morgan folds), cheilitis, recurrent conjunctivitis, anterior neck folds.
Triggers: foods, emotional factors, environmental factors, skin irritants.
Complications: susceptibility to cutaneous infections, impaired cell-mediated immunity, immediate skin-test reactivity, elevated IgE, keratoconus, anterior subcapsular cataracts.
Other: early age of onset, dry skin, ichthyosis, hyperlinear palms, keratosis pilaris, hand and foot dermatitis, nipple eczema, white dermatographism, perifollicular accentuation.

### Example 3

The objective of this double-blinded Phase 2b study was to assess the efficacy and safety of orally applied DS107 to adults with moderate to severe AD. The study used a randomized (1:1:1, Treatment Group A: 1 g DS107, Treatment Group B: 2 g DS107 and Treatment Group C: Placebo), double-blind, placebo-controlled parallel group design as described in Example 2. Disposition, compliance, demographics, and primary and secondary efficacy results are presented by treatment group separately for moderate and severe AD as well as for combined groups.

Blood samples of subjects in the Per-Protocol Set (PPS), collected via direct venepuncture at Baseline/Visit 2 and Week 8/Visit 6, were evaluated for Atopic Dermatitis biomarkers using the OLINK high-throughput proteomics platform. The PPS is the subset of the Full Analysis Set (FAS) who completed the study without any major violations.

Each patient sample at Baseline and Week 8 as described above in Example 2 were analyzed using the OLINK high-throughput proteomic platform, to quantify the panels of proteins (i.e., biomarker analytes) listed in Table 16.

TABLE 16
OLINK Proteomic Biomarker Panels and Analytes

| Panel | Assay | Uniprot ID |
|---|---|---|
| Olink CARDIOVASCULAR II | BMP-6 | P22004 |
| Olink CARDIOVASCULAR II | SRC | P12931 |
| Olink CARDIOVASCULAR II | IL-1ra | P18510 |
| Olink CARDIOVASCULAR II | IL6 | P05231 |
| Olink CARDIOVASCULAR II | TNFRSF10A | O00220 |
| Olink CARDIOVASCULAR II | STK4 | Q13043 |
| Olink CARDIOVASCULAR II | IDUA | P35475 |
| Olink CARDIOVASCULAR II | TNFRSF11A | Q9Y6Q6 |
| Olink CARDIOVASCULAR II | PAR-1 | P25116 |
| Olink CARDIOVASCULAR II | TRAIL-R2 | O14763 |
| Olink CARDIOVASCULAR II | PRSS27 | Q9BQR3 |
| Olink CARDIOVASCULAR II | ANG-1 | Q15389 |
| Olink CARDIOVASCULAR II | TIE2 | Q02763 |
| Olink CARDIOVASCULAR II | TF | P13726 |
| Olink CARDIOVASCULAR II | IL1RL2 | Q9HB29 |
| Olink CARDIOVASCULAR II | PDGF subunit B | P01127 |
| Olink CARDIOVASCULAR II | IL-27 | Q8NEV9, |
| | | Q14213 |
| Olink CARDIOVASCULAR II | IL-17D | Q8TAD2 |
| Olink CARDIOVASCULAR II | CXCL1 | P09341 |
| Olink CARDIOVASCULAR II | LOX-1 | P78380 |
| Olink CARDIOVASCULAR II | Gal-9 | O00182 |
| Olink CARDIOVASCULAR II | GIF | P27352 |
| Olink CARDIOVASCULAR II | ADM | P35318 |
| Olink CARDIOVASCULAR II | SCF | P21583 |
| Olink CARDIOVASCULAR II | IL18 | Q14116 |
| Olink CARDIOVASCULAR II | FGF-21 | Q9NSA1 |
| Olink CARDIOVASCULAR II | PIgR | P01833 |
| Olink CARDIOVASCULAR II | RAGE | Q15109 |
| Olink CARDIOVASCULAR II | SOD2 | P04179 |
| Olink CARDIOVASCULAR II | CTRC | Q99895 |
| Olink CARDIOVASCULAR II | FGF-23 | Q9GZV9 |
| Olink CARDIOVASCULAR II | SPON2 | Q9BUD6 |
| Olink CARDIOVASCULAR II | GH | P01241 |
| Olink CARDIOVASCULAR II | CD40-L | P29965 |
| Olink CARDIOVASCULAR II | FS | P19883 |
| Olink CARDIOVASCULAR II | GLO1 | Q04760 |
| Olink CARDIOVASCULAR II | CD84 | Q9UIB8 |
| Olink CARDIOVASCULAR II | PAPPA | Q13219 |
| Olink CARDIOVASCULAR II | SERPINA12 | Q8IW75 |
| Olink CARDIOVASCULAR II | REN | P00797 |
| Olink CARDIOVASCULAR II | DECR1 | Q16698 |
| Olink CARDIOVASCULAR II | MERTK | Q12866 |
| Olink CARDIOVASCULAR II | KIMI | Q96D42 |
| Olink CARDIOVASCULAR II | THBS2 | P35442 |
| Olink CARDIOVASCULAR II | SLAMF7 | Q9NQ25 |
| Olink CARDIOVASCULAR II | TM | P07204 |
| Olink CARDIOVASCULAR II | VSIG2 | Q96IQ7 |
| Olink CARDIOVASCULAR II | AMBP | P02760 |
| Olink CARDIOVASCULAR II | PRELP | P51888 |
| Olink CARDIOVASCULAR II | HO-1 | P09601 |
| Olink CARDIOVASCULAR II | XCL1 | P47992 |
| Olink CARDIOVASCULAR II | IL16 | Q14005 |
| Olink CARDIOVASCULAR II | SORT1 | Q99523 |
| Olink CARDIOVASCULAR II | CEACAM8 | P31997 |
| Olink CARDIOVASCULAR II | PTX3 | P26022 |
| Olink CARDIOVASCULAR II | PGF | P49763 |
| Olink CARDIOVASCULAR II | PSGL-1 | Q14242 |
| Olink CARDIOVASCULAR II | CCL17 | Q92583 |
| Olink CARDIOVASCULAR II | CCL-3 | P10147 |
| Olink CARDIOVASCULAR II | MMP7 | P09237 |
| Olink CARDIOVASCULAR II | IgG Fc receptor II-b | P31994 |
| Olink CARDIOVASCULAR II | ITGB1BP2 | Q9UKP3 |
| Olink CARDIOVASCULAR II | DCN | P07585 |
| Olink CARDIOVASCULAR II | Dkk-1 | O94907 |
| Olink CARDIOVASCULAR II | LPL | P06858 |
| Olink CARDIOVASCULAR II | PRSS8 | Q16651 |
| Olink CARDIOVASCULAR II | ADAM-TS13 | Q76LX8 |
| Olink CARDIOVASCULAR II | AGRP | O00253 |
| Olink CARDIOVASCULAR II | HB-EGF | Q99075 |
| Olink CARDIOVASCULAR II | GDF-2 | Q9UK05 |
| Olink CARDIOVASCULAR II | FABP2 | P12104 |
| Olink CARDIOVASCULAR II | THPO | P40225 |
| Olink CARDIOVASCULAR II | MARCO | Q9UEW3 |
| Olink CARDIOVASCULAR II | GT | P51161 |
| Olink CARDIOVASCULAR II | BNP | P16860 |
| Olink CARDIOVASCULAR II | MMP12 | P39900 |
| Olink CARDIOVASCULAR II | ACE2 | Q9BYF1 |
| Olink CARDIOVASCULAR II | BOC | Q9BWV1 |
| Olink CARDIOVASCULAR II | PD-L2 | Q9BQ51 |
| Olink CARDIOVASCULAR II | CTSL1 | P07711 |
| Olink CARDIOVASCULAR II | hOSCAR | Q8IYS5 |
| Olink CARDIOVASCULAR II | TNFRSF13B | O14836 |
| Olink CARDIOVASCULAR II | TGM2 | P21980 |
| Olink CARDIOVASCULAR II | LEP | P41159 |
| Olink CARDIOVASCULAR II | CA5A | P35218 |
| Olink CARDIOVASCULAR II | HSP 27 | P04792 |
| Olink CARDIOVASCULAR II | CD4 | P01730 |
| Olink CARDIOVASCULAR II | NEMO | Q9Y6K9 |
| Olink CARDIOVASCULAR II | IL-4RA | P24394 |
| Olink CARDIOVASCULAR II | VEGFD | O43915 |
| Olink CARDIOVASCULAR II | PARP-1 | P09874 |
| Olink CARDIOVASCULAR II | HAOX1 | Q9UJM8 |
| Olink CARDIOVASCULAR III | TNFRSF14 | Q92956 |
| Olink CARDIOVASCULAR III | ALCAM | Q13740 |
| Olink CARDIOVASCULAR III | TFF3 | Q07654 |
| Olink CARDIOVASCULAR III | SELP | P16109 |
| Olink CARDIOVASCULAR III | CSTB | P04080 |
| Olink CARDIOVASCULAR III | MCP-1 | P13500 |
| Olink CARDIOVASCULAR III | CD163 | Q86VB7 |
| Olink CARDIOVASCULAR III | Gal-3 | P17931 |
| Olink CARDIOVASCULAR III | GRN | P28799 |
| Olink CARDIOVASCULAR III | NT-proBNP | NA |
| Olink CARDIOVASCULAR III | BLM hydrolase | Q13867 |
| Olink CARDIOVASCULAR III | LDL receptor | P01130 |
| Olink CARDIOVASCULAR III | PLC | P98160 |
| Olink CARDIOVASCULAR III | LTBR | P36941 |
| Olink CARDIOVASCULAR III | Notch 3 | Q9UM47 |
| Olink CARDIOVASCULAR III | TIMP4 | Q99727 |
| Olink CARDIOVASCULAR III | CNTN1 | Q12860 |
| Olink CARDIOVASCULAR III | CDH5 | P33151 |
| Olink CARDIOVASCULAR III | TLT-2 | Q5T2D2 |
| Olink CARDIOVASCULAR III | FABP4 | P15090 |
| Olink CARDIOVASCULAR III | TFPI | P10646 |
| Olink CARDIOVASCULAR III | PAI | P05121 |
| Olink CARDIOVASCULAR III | ITGB2 | P05107 |
| Olink CARDIOVASCULAR III | CCL-24 | O00175 |
| Olink CARDIOVASCULAR III | TR | P02786 |
| Olink CARDIOVASCULAR III | TNFRSF10C | O14798 |
| Olink CARDIOVASCULAR III | GDF-15 | Q99988 |
| Olink CARDIOVASCULAR III | SELE | P16581 |
| Olink CARDIOVASCULAR III | AZU1 | P20160 |
| Olink CARDIOVASCULAR III | DLK-1 | P80370 |
| Olink CARDIOVASCULAR III | SPON1 | Q9HCB6 |
| Olink CARDIOVASCULAR III | MPO | P05164 |
| Olink CARDIOVASCULAR III | CXCL16 | Q9H2A7 |
| Olink CARDIOVASCULAR III | IL-17RA | Q96F46 |
| Olink CARDIOVASCULAR III | IL-6RA | P08887 |
| Olink CARDIOVASCULAR III | RETN | Q9HD89 |
| Olink CARDIOVASCULAR III | IGFBP-1 | P08833 |
| Olink CARDIOVASCULAR III | CHIT1 | Q13231 |
| Olink CARDIOVASCULAR III | TR-AP | P13686 |
| Olink CARDIOVASCULAR III | PSP-D | P35247 |
| Olink CARDIOVASCULAR III | PI3 | P19957 |
| Olink CARDIOVASCULAR III | Ep-CAM | P16422 |
| Olink CARDIOVASCULAR III | AP-N | P15144 |
| Olink CARDIOVASCULAR III | TNF-R2 | P20333 |
| Olink CARDIOVASCULAR III | AXL | P30530 |
| Olink CARDIOVASCULAR III | IL-1RT1 | P14778 |
| Olink CARDIOVASCULAR III | MMP-2 | P08253 |
| Olink CARDIOVASCULAR III | FAS | P25445 |
| Olink CARDIOVASCULAR III | MB | P02144 |
| Olink CARDIOVASCULAR III | TNFSF13B | Q9Y275 |
| Olink CARDIOVASCULAR III | PRTN3 | P24158 |
| Olink CARDIOVASCULAR III | PCSK9 | Q8NBP7 |
| Olink CARDIOVASCULAR III | U-PAR | Q03405 |
| Olink CARDIOVASCULAR III | OPN | P10451 |
| Olink CARDIOVASCULAR III | MMP-9 | P14780 |
| Olink CARDIOVASCULAR III | CTSD | P07339 |
| Olink CARDIOVASCULAR III | PGLYRP1 | O75594 |
| Olink CARDIOVASCULAR III | CPA1 | P15085 |
| Olink CARDIOVASCULAR III | JAM-A | Q9Y624 |
| Olink CARDIOVASCULAR III | Gal-4 | P56470 |
| Olink CARDIOVASCULAR III | IL-1RT2 | P27930 |
| Olink CARDIOVASCULAR III | SHPS-1 | P78324 |
| Olink CARDIOVASCULAR III | CCL-15 | Q16663 |
| Olink CARDIOVASCULAR III | CASP-3 | P42574 |
| Olink CARDIOVASCULAR III | uPA | P00749 |
| Olink CARDIOVASCULAR III | EPHB4 | P54760 |
| Olink CARDIOVASCULAR III | CPB1 | P15086 |
| Olink CARDIOVASCULAR III | CHI3L1 | P36222 |
| Olink CARDIOVASCULAR III | ST2 | Q01638 |
| Olink CARDIOVASCULAR III | t-PA | P00750 |
| Olink CARDIOVASCULAR III | SCGB3A2 | Q96PL1 |
| Olink CARDIOVASCULAR III | EGFR | P00533 |
| Olink CARDIOVASCULAR III | IGFBP-7 | Q16270 |
| Olink CARDIOVASCULAR III | CD93 | Q9NPY3 |
| Olink CARDIOVASCULAR III | IL-18BP | O95998 |
| Olink CARDIOVASCULAR III | COL1A1 | P02452 |
| Olink CARDIOVASCULAR III | IL2-RA | P01589 |
| Olink CARDIOVASCULAR III | PON3 | Q15166 |
| Olink CARDIOVASCULAR III | CTSZ | Q9UBR2 |
| Olink CARDIOVASCULAR III | MMP-3 | P08254 |
| Olink CARDIOVASCULAR III | RARRES2 | Q99969 |
| Olink CARDIOVASCULAR III | ICAM-2 | P13598 |
| Olink CARDIOVASCULAR III | KLK6 | Q92876 |
| Olink CARDIOVASCULAR III | PDGF subunit A | P04085 |
| Olink CARDIOVASCULAR III | TNF-R1 | P19438 |
| Olink CARDIOVASCULAR III | IGFBP-2 | P18065 |
| Olink CARDIOVASCULAR III | vWF | P04275 |
| Olink CARDIOVASCULAR III | OPG | O00300 |
| Olink CARDIOVASCULAR III | PECAM-1 | P16284 |
| Olink CARDIOVASCULAR III | MEPE | Q9NQ76 |
| Olink CARDIOVASCULAR III | CCL-16 | O15467 |
| Olink INFLAMMATION | IL8 | P10145 |
| Olink INFLAMMATION | uPA | P00749 |
| Olink INFLAMMATION | IL6 | P05231 |
| Olink INFLAMMATION | IL-17C | Q9P0M4 |
| Olink INFLAMMATION | MCP-1 | P13500 |
| Olink INFLAMMATION | IL-17A | Q16552 |
| Olink INFLAMMATION | CXCL11 | O14625 |
| Olink INFLAMMATION | AXIN1 | O15169 |
| Olink INFLAMMATION | TRAIL | P50591 |
| Olink INFLAMMATION | IL-20RA | Q9UHF4 |
| Olink INFLAMMATION | CXCL9 | Q07325 |
| Olink INFLAMMATION | VEGF-A | P15692 |
| Olink INFLAMMATION | CST5 | P28325 |
| Olink INFLAMMATION | IL-2RB | P14784 |
| Olink INFLAMMATION | IL-1 alpha | P01583 |
| Olink INFLAMMATION | OSM | P13725 |
| Olink INFLAMMATION | IL2 | P60568 |
| Olink INFLAMMATION | CXCL1 | P09341 |
| Olink INFLAMMATION | TSLP | Q969D9 |
| Olink INFLAMMATION | CCL-4 | P13236 |
| Olink INFLAMMATION | CD6 | Q8WWJ7 |
| Olink INFLAMMATION | SCF | P21583 |
| Olink INFLAMMATION | MCP-3 | P80098 |
| Olink INFLAMMATION | IL18 | Q14116 |
| Olink INFLAMMATION | SLAMF1 | Q13291 |
| Olink INFLAMMATION | TGF-alpha | P01135 |
| Olink INFLAMMATION | MCP-4 | Q99616 |
| Olink INFLAMMATION | CCL-11 | P51671 |
| Olink INFLAMMATION | TNFSF14 | O43557 |
| Olink INFLAMMATION | FGF-23 | Q9GZV9 |
| Olink INFLAMMATION | IL-10RA | Q13651 |
| Olink INFLAMMATION | FGF-5 | Q8NF90 |
| Olink INFLAMMATION | MMP-1 | P03956 |
| Olink INFLAMMATION | GDNF | P39905 |
| Olink INFLAMMATION | LIF-R | P42702 |
| Olink INFLAMMATION | FGF-21 | Q9NSA1 |
| Olink INFLAMMATION | CCL-19 | Q99731 |
| Olink INFLAMMATION | IL-15RA | Q13261 |
| Olink INFLAMMATION | IL-10RB | Q08334 |
| Olink INFLAMMATION | IL-22 RA1 | Q8N6P7 |
| Olink INFLAMMATION | IL-18R1 | Q13478 |
| Olink INFLAMMATION | PD-L1 | Q9NZQ7 |
| Olink INFLAMMATION | Beta-NGF | P01138 |
| Olink INFLAMMATION | CXCL5 | P42830 |
| Olink INFLAMMATION | CDCP1 | Q9H5V8 |
| Olink INFLAMMATION | TRANCE | O14788 |
| Olink INFLAMMATION | HGF | P14210 |
| Olink INFLAMMATION | IL-12B | P29460 |
| Olink INFLAMMATION | IL-24 | Q13007 |
| Olink INFLAMMATION | IL13 | P35225 |
| Olink INFLAMMATION | ARTN | Q5T4W7 |
| Olink INFLAMMATION | MMP-10 | P09238 |
| Olink INFLAMMATION | IL10 | P22301 |
| Olink INFLAMMATION | TNF | P01375 |
| Olink INFLAMMATION | CCL-23 | P55773 |
| Olink INFLAMMATION | CD244 | Q9BZW8 |
| Olink INFLAMMATION | CD5 | P06127 |
| Olink INFLAMMATION | CCL-3 | P10147 |
| Olink INFLAMMATION | Flt3L | P49771 |
| Olink INFLAMMATION | CXCL6 | P80162 |
| Olink INFLAMMATION | CXCL10 | P02778 |
| Olink INFLAMMATION | 4E-BP1 | Q13541 |
| Olink INFLAMMATION | IL-20 | Q9NYY1 |
| Olink INFLAMMATION | SIRT2 | Q8IXJ6 |
| Olink INFLAMMATION | CCL-28 | Q9NRJ3 |
| Olink INFLAMMATION | DNER | Q8NFT8 |
| Olink INFLAMMATION | IL7 | P13232 |
| Olink INFLAMMATION | EN-RAGE | P80511 |
| Olink INFLAMMATION | CD40 | P25942 |
| Olink INFLAMMATION | IL33 | O95760 |
| Olink INFLAMMATION | IFN-gamma | P01579 |
| Olink INFLAMMATION | FGF-19 | O95750 |
| Olink INFLAMMATION | IL4 | P05112 |
| Olink INFLAMMATION | LIF | P15018 |
| Olink INFLAMMATION | NRTN | Q99748 |
| Olink INFLAMMATION | MCP-2 | P80075 |
| Olink INFLAMMATION | CASP-8 | Q14790 |
| Olink INFLAMMATION | OPG | O00300 |
| Olink INFLAMMATION | CCL-25 | O15444 |
| Olink INFLAMMATION | CX3CL1 | P78423 |
| Olink INFLAMMATION | TNFRSF9 | Q07011 |
| Olink INFLAMMATION | NT-3 | P20783 |
| Olink INFLAMMATION | TWEAK | O43508 |
| Olink INFLAMMATION | CCL-20 | P78556 |
| Olink INFLAMMATION | ST1A1 | P50225 |
| Olink INFLAMMATION | STAMPB | O95630 |
| Olink INFLAMMATION | IL5 | P05113 |
| Olink INFLAMMATION | ADA | P00813 |
| Olink INFLAMMATION | LAP TGF-beta-1 | P01137 |
| Olink INFLAMMATION | TNFB | P01374 |
| Olink INFLAMMATION | CSF-1 | P09603 |
| Olink NEUROLOGY | NMNAT1 | Q9HAN9 |
| Olink NEUROLOGY | SMOC2 | Q9H3U7 |
| Olink NEUROLOGY | NBL1 | P41271 |
| Olink NEUROLOGY | EFNA4 | P52798 |
| Olink NEUROLOGY | SCARB2 | Q14108 |
| Olink NEUROLOGY | NCAN | O14594 |
| Olink NEUROLOGY | PRTG | Q2VWP7 |
| Olink NEUROLOGY | ROBO2 | Q9HCK4 |
| Olink NEUROLOGY | CRTAM | O95727 |
| Olink NEUROLOGY | RGMA | Q96B86 |
| Olink NEUROLOGY | PLXNB3 | Q9ULL4 |
| Olink NEUROLOGY | NRP2 | O60462 |
| Olink NEUROLOGY | CPA2 | P48052 |
| Olink NEUROLOGY | CD38 | P28907 |
| Olink NEUROLOGY | SMPD1 | P17405 |
| Olink NEUROLOGY | MSR1 | P21757 |
| Olink NEUROLOGY | Alpha-2-MRAP | P30533 |
| Olink NEUROLOGY | sFRP-3 | Q92765 |
| Olink NEUROLOGY | EPHB6 | O15197 |
| Olink NEUROLOGY | RGMB | Q6NW40 |
| Olink NEUROLOGY | SIGLEC1 | Q9BZZ2 |
| Olink NEUROLOGY | CNTN5 | O94779 |
| Olink NEUROLOGY | CADM3 | Q8N126 |
| Olink NEUROLOGY | ADAM 22 | Q9P0K1 |
| Olink NEUROLOGY | CLEC1B | Q9P126 |
| Olink NEUROLOGY | ADAM 23 | O75077 |
| Olink NEUROLOGY | MATN3 | O15232 |
| Olink NEUROLOGY | RSPO1 | Q2MKA7 |
| Olink NEUROLOGY | HAGH | Q16775 |
| Olink NEUROLOGY | LXN | Q9BS40 |
| Olink NEUROLOGY | gal-8 | O00214 |
| Olink NEUROLOGY | BCAN | Q96GW7 |
| Olink NEUROLOGY | LAYN | Q6UX15 |
| Olink NEUROLOGY | GDNF | P39905 |
| Olink NEUROLOGY | NEP | P08473 |
| Olink NEUROLOGY | GDF-8 | O14793 |
| Olink NEUROLOGY | THY 1 | P04216 |
| Olink NEUROLOGY | WFIKKN1 | Q96NZ8 |
| Olink NEUROLOGY | TMPRSS5 | Q9H3S3 |
| Olink NEUROLOGY | CDH3 | P22223 |
| Olink NEUROLOGY | GFR-alpha-1 | P56159 |
| Olink NEUROLOGY | GM-CSF-R-alpha | P15509 |
| Olink NEUROLOGY | Beta-NGF | P01138 |
| Olink NEUROLOGY | SCARA5 | Q6ZMJ2 |
| Olink NEUROLOGY | UNC5C | O95185 |
| Olink NEUROLOGY | CD200 | P41217 |
| Olink NEUROLOGY | NTRK2 | Q16620 |
| Olink NEUROLOGY | GZMA | P12544 |
| Olink NEUROLOGY | G-CSF | P09919 |
| Olink NEUROLOGY | DRAXIN | Q8NBI3 |
| Olink NEUROLOGY | SCARF2 | Q96GP6 |
| Olink NEUROLOGY | GDNFR-alpha-3 | O60609 |
| Olink NEUROLOGY | PVR | P15151 |
| Olink NEUROLOGY | TNFRSF12A | Q9NP84 |
| Olink NEUROLOGY | SKR3 | P37023 |
| Olink NEUROLOGY | VWC2 | Q2TAL6 |
| Olink NEUROLOGY | FLRT2 | O43155 |
| Olink NEUROLOGY | CPM | P14384 |
| Olink NEUROLOGY | CLEC10A | Q8IUN9 |
| Olink NEUROLOGY | GCP5 | P78333 |
| Olink NEUROLOGY | BMP-4 | P12644 |
| Olink NEUROLOGY | FcRL2 | Q96LA5 |
| Olink NEUROLOGY | MDGA1 | Q8NFP4 |
| Olink NEUROLOGY | IL-5R-alpha | Q01344 |
| Olink NEUROLOGY | PDGF-R-alpha | P16234 |
| Olink NEUROLOGY | CTSC | P53634 |
| Olink NEUROLOGY | Siglec-9 | Q9Y336 |
| Olink NEUROLOGY | CDH6 | P55285 |
| Olink NEUROLOGY | DDR1 | Q08345 |
| Olink NEUROLOGY | JAM-B | P57087 |
| Olink NEUROLOGY | CTSS | P25774 |
| Olink NEUROLOGY | N-CDase | Q9NR71 |
| Olink NEUROLOGY | NAAA | Q02083 |
| Olink NEUROLOGY | N2DL-2 | Q9BZM5 |
| Olink NEUROLOGY | PLXNB1 | O43157 |
| Olink NEUROLOGY | TNFRSF21 | O75509 |
| Olink NEUROLOGY | CLM-1 | Q8TDQ1 |
| Olink NEUROLOGY | CLM-6 | Q08708 |
| Olink NEUROLOGY | SPOCK1 | Q08629 |
| Olink NEUROLOGY | IL12 | P29460, |
| | | P29459 |
| Olink NEUROLOGY | Dkk-4 | Q9UBT3 |
| Olink NEUROLOGY | EDA2R | Q9HAV5 |
| Olink NEUROLOGY | LAT | O43561 |
| Olink NEUROLOGY | NTRK3 | Q16288 |
| Olink NEUROLOGY | LAIR-2 | Q6ISS4 |
| Olink NEUROLOGY | MANF | P55145 |
| Olink NEUROLOGY | TN-R | Q92752 |
| Olink NEUROLOGY | CD200R1 | Q8TD46 |
| Olink NEUROLOGY | EZR | P15311 |
| Olink NEUROLOGY | Nr-CAM | Q92823 |
| Olink NEUROLOGY | KYNU | Q16719 |

Demographics and Other Baseline Characteristics: Descriptive statistics of demographics and other baseline characteristics are presented for all subjects and tabulated by treatment group. Baseline characteristics include age, sex, ethnicity, race, IGA and Pruritus NRS.

General Statistical Methods: Four panels of OLINK analytes were evaluated, including inflammatory markers that are generally analyzed in skin such as Th2 chemokines, Th1 chemokines, as well as cardiovascular markers, neuro-immunological markers, and many other markers. Blood assessments were performed on serum obtained at baseline, and after treatment. OLINK normalized protein expression (NPX) values in log 2 scale were modeled using a linear mixed effect model with Time and Treatment as a fixed effect and a random intercept for each patient. This formulation intrinsically models the within patient correlation structure as in the case of a paired t-test. The mixed-effect model has the advantage that estimation of the parameters was possible even in the presence of missing values. This approach introduces less bias than restricting the analysis for patients with complete observations. Contrasts were used to estimate the fold changes with treatment within each treatment group and to conduct hypothesis testing. P-values were adjusted for multiple hypotheses using the Benjamini-Hochberg procedure, which controls for False Discovery Rate (FDR).

Correlations with disease severity: Pearson and Spearman correlation coefficients between baseline clinical data (IGA, NRS) and biomarker levels were computed. In addition, correlations of post vs. pre-treatment changes between clinical data and biomarker levels were provided for each treatment group.

Statistical outputs for this study were generated for the following:
Differentially expressed markers between placebo and 2 g DS107 and 1 g DS107 active at each time point; baseline and Week 8/end of treatment (EOT) including: Fold-changes between active and placebo, p-values, and FDR values.
Subgroup analysis of differentially expressed markers between placebo and 2 g DS107 and 1 g DS107 active at each time point in IGA responder and Non-responders including: Fold-changes between active and placebo, p-values, and FDR values.
Barplots to visualize the fold-changes of each treatment group vs. baseline, including sub-group analysis.
Correlations between baseline clinical data (IGA, NRS) and baseline biomarker levels for each treatment group including: Correlation coefficients (Pearson, Spearman), p-values, FDR values, and scatter plots.
Correlation of baseline biomarker levels and changes in clinical data (IGA responders at Week 8/EOT, change in pruritus NRS from baseline to Week 8/EOT) for each treatment group including: Correlation coefficients (Pearson, Spearman), p-values, FDR values, and scatter plots.
Correlations between changes in clinical data (IGA response at Week 8/EOT, change in pruritus NRS from baseline to Week 8/EOT) and changes in biomarker levels for each treatment group including: Correlation coefficients (Pearson, Spearman), p-values, FDR values, and scatter plots.

Results

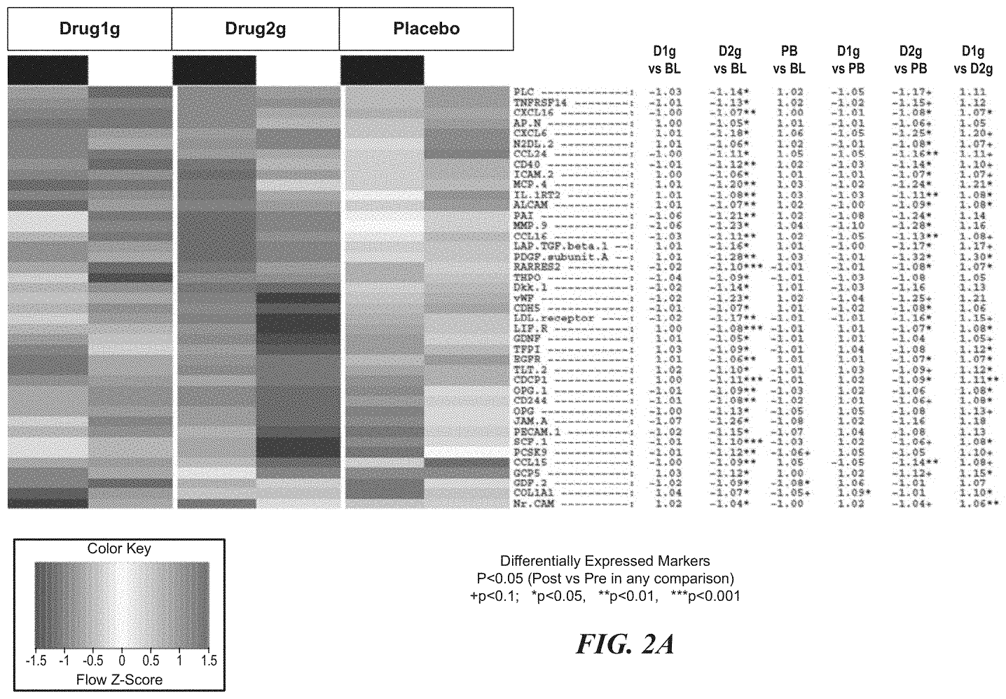

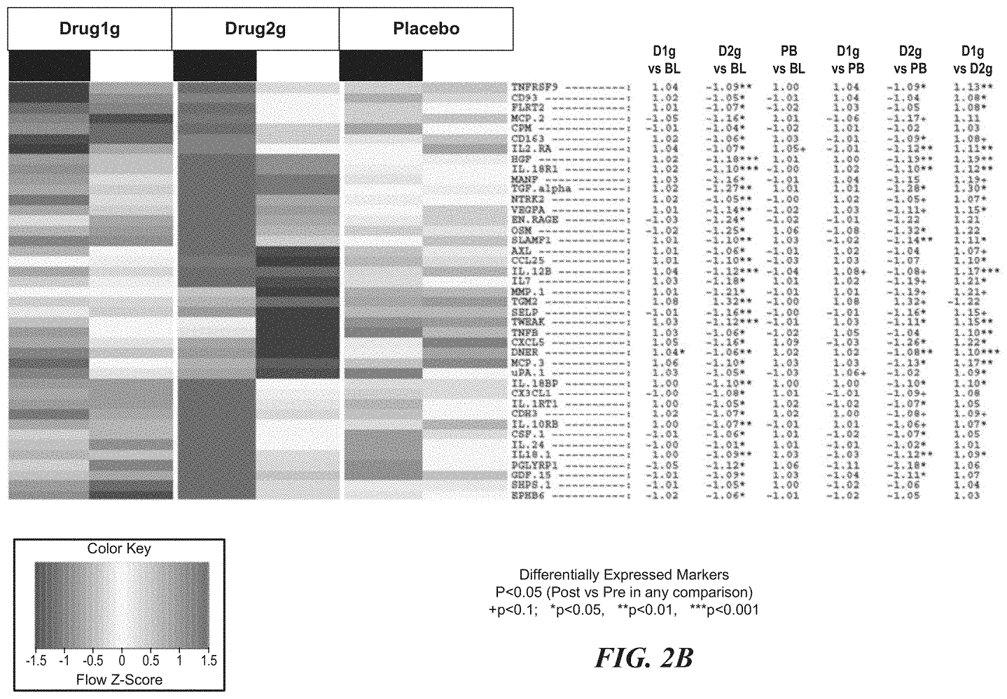

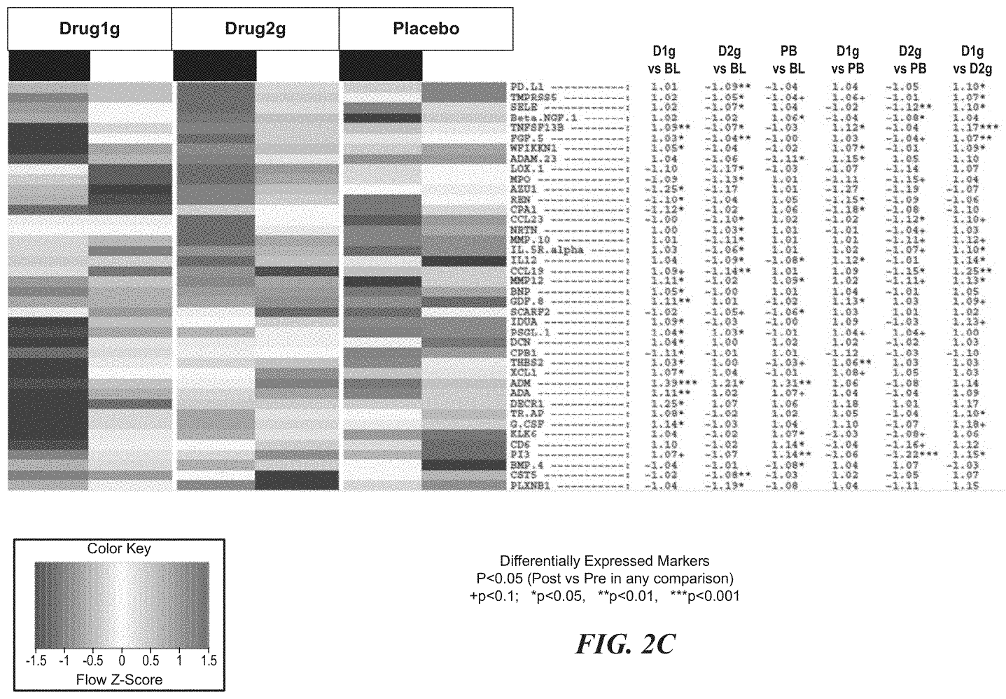

Differentially Expressed Biomarkers: FIGS. 2A-2C are heat maps showing the mean abundance of the differentially expressed biomarkers for each treatment group where Drug1g refers to Treatment Group A: 1 g DS107, Drug2g refers to Treatment Group B: 2 g DS107 and Placebo refers to Treatment Group C: placebo. FIGS. 2A-2C show that 122 different biomarker levels were measured and then compared from baseline (BL) to end of treatment (EOT) based in patients based on each treatment group. Importantly, patients from Treatment Group A (i.e., Drug2g) who were administered 2 g of DS107 daily for 8 weeks exhibited molecular improvement from end of treatment as compared to baseline.

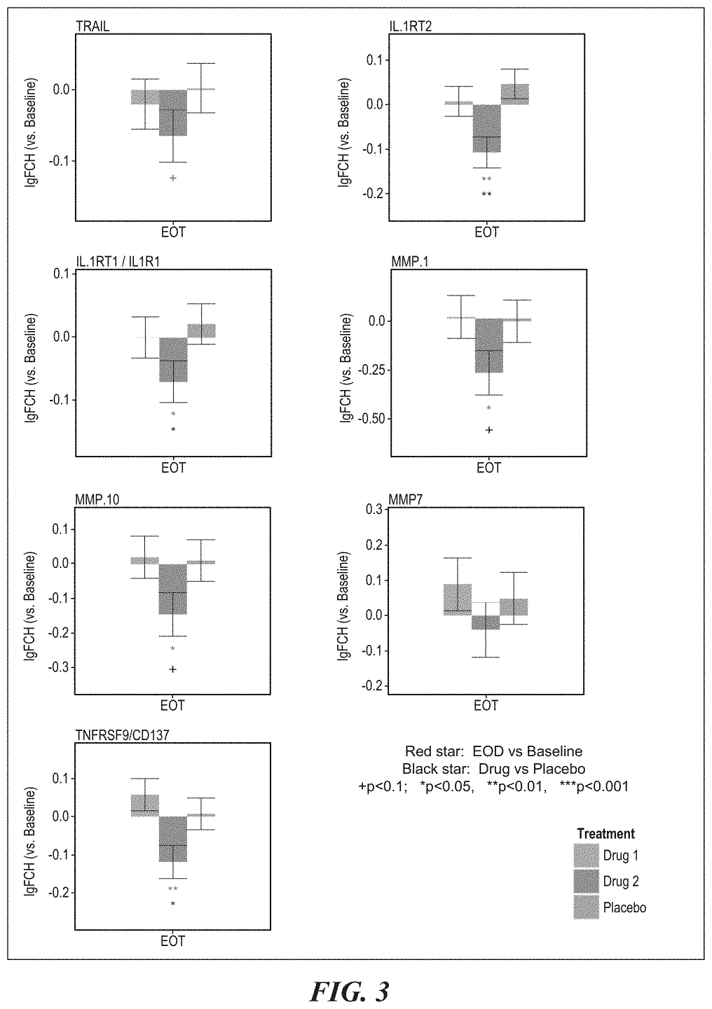

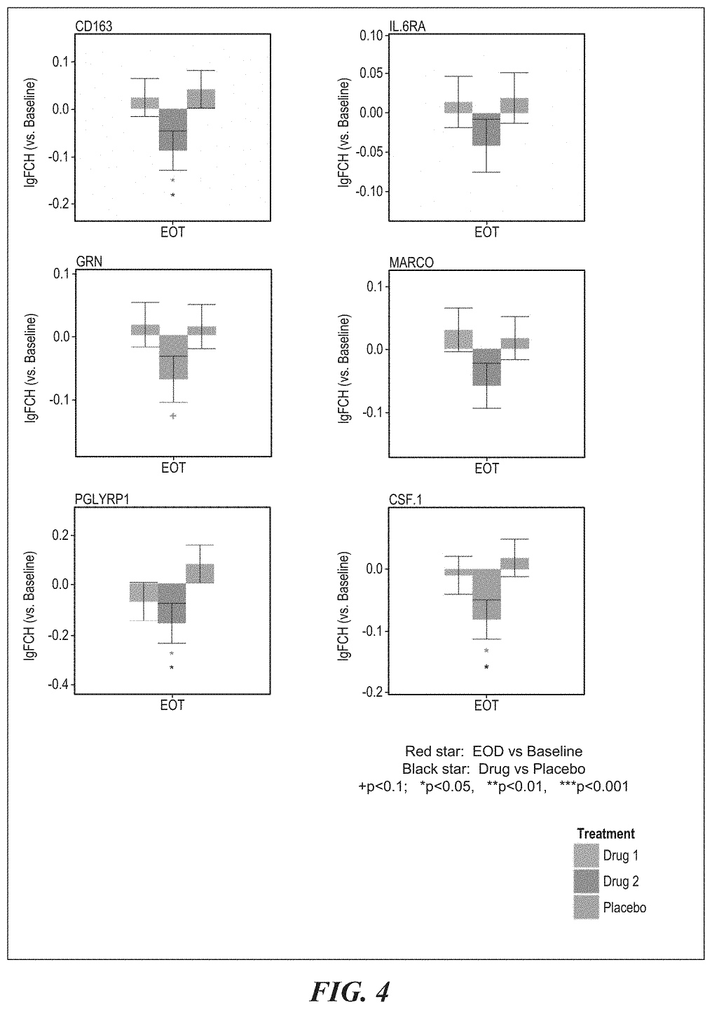

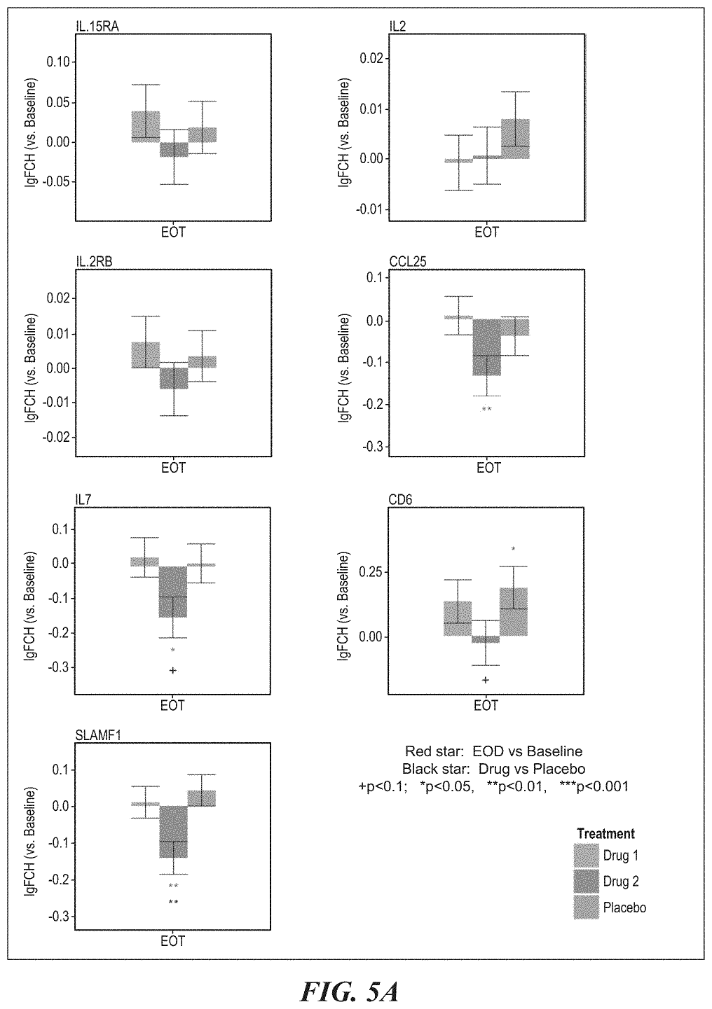

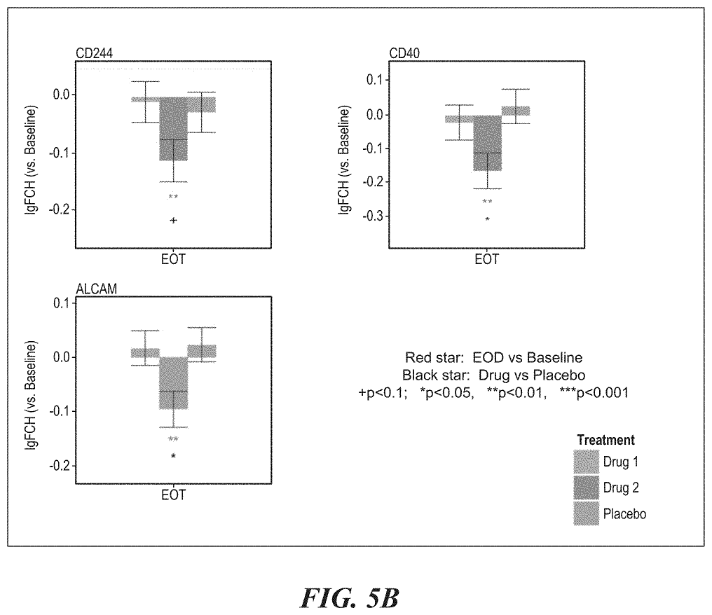

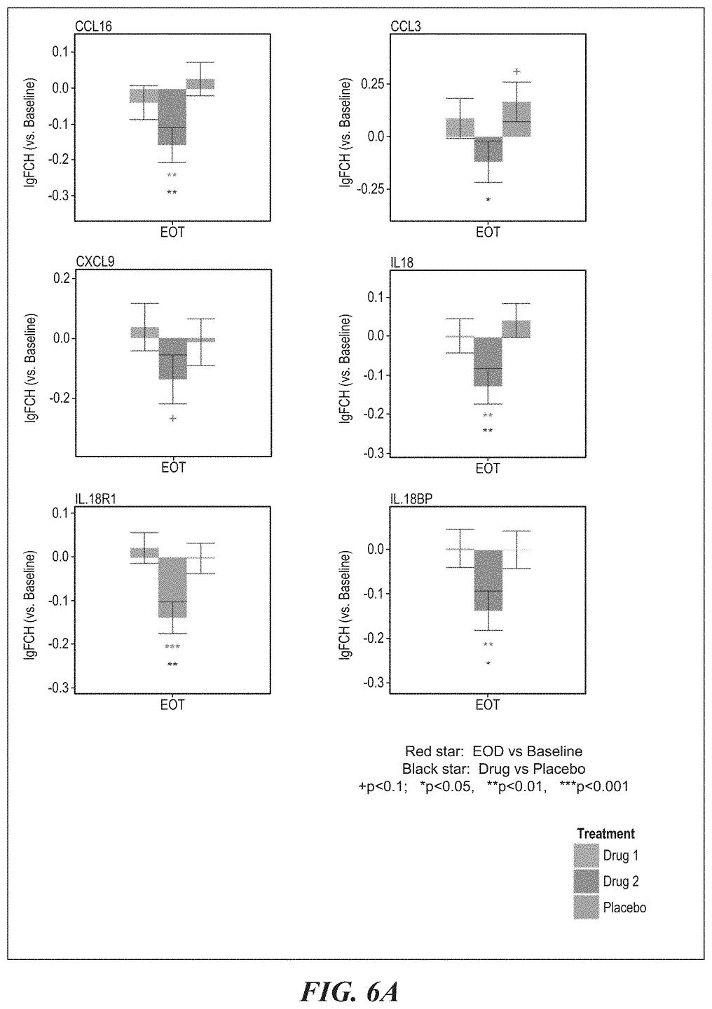

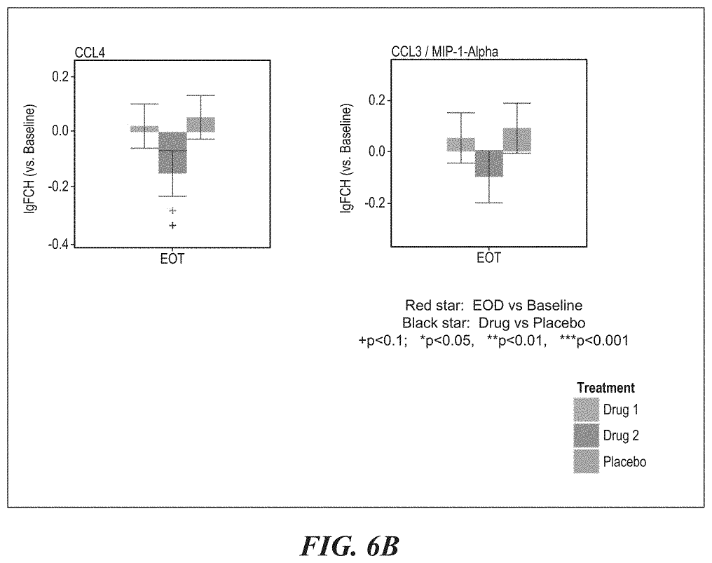

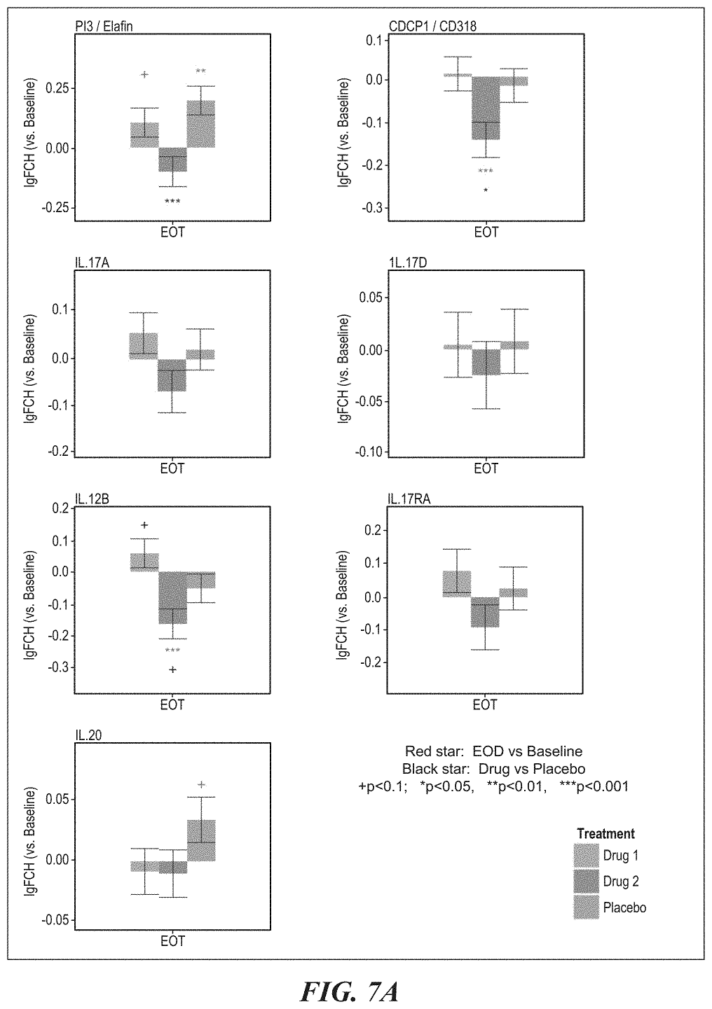

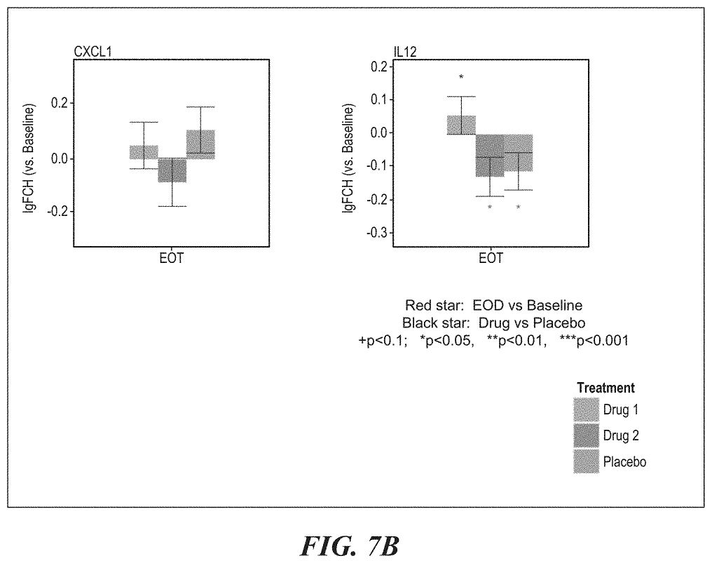

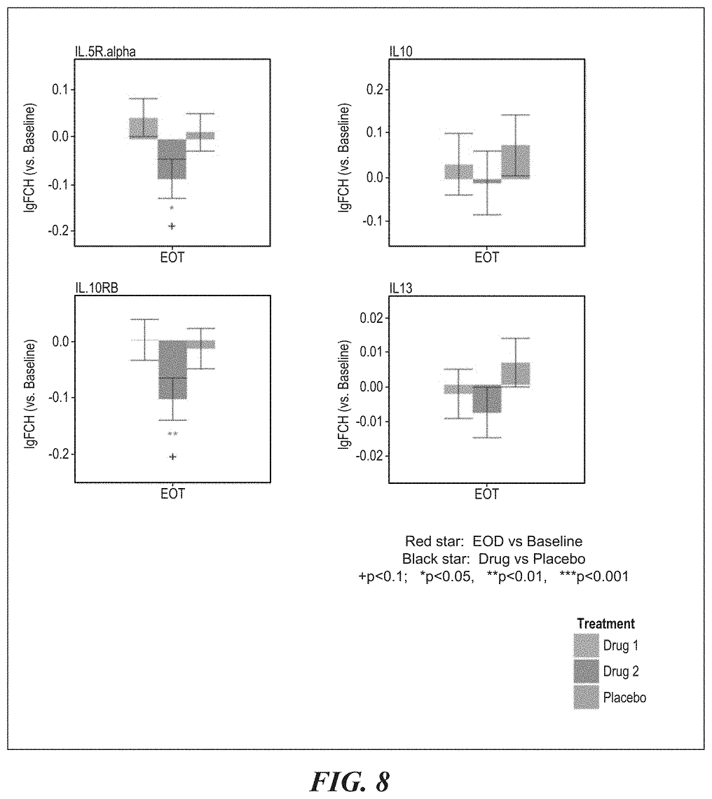

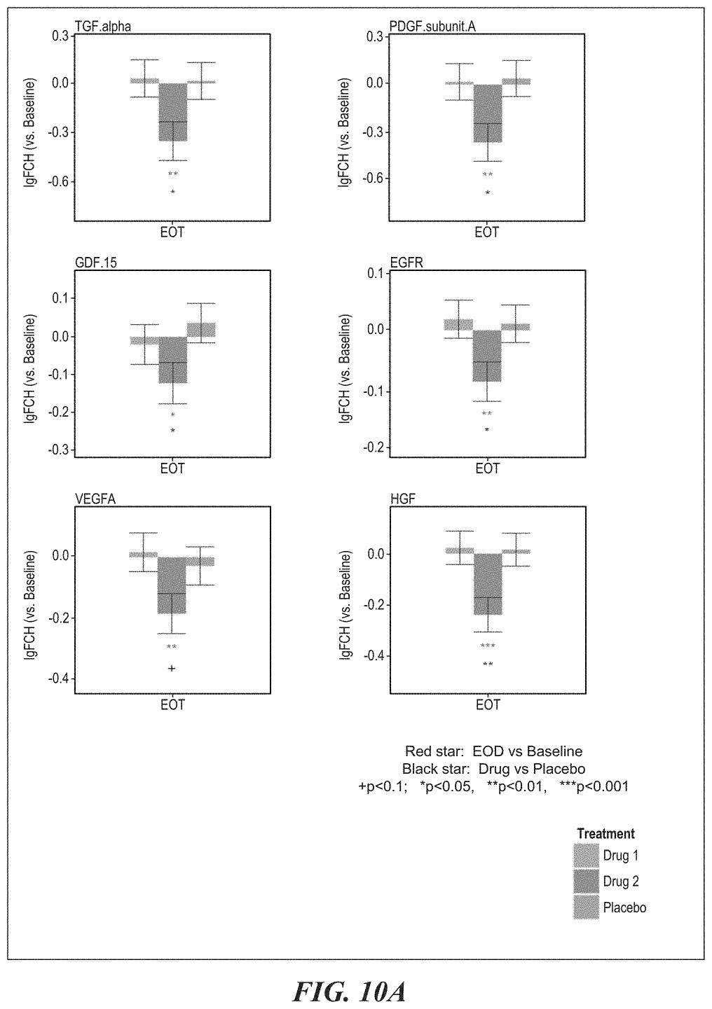

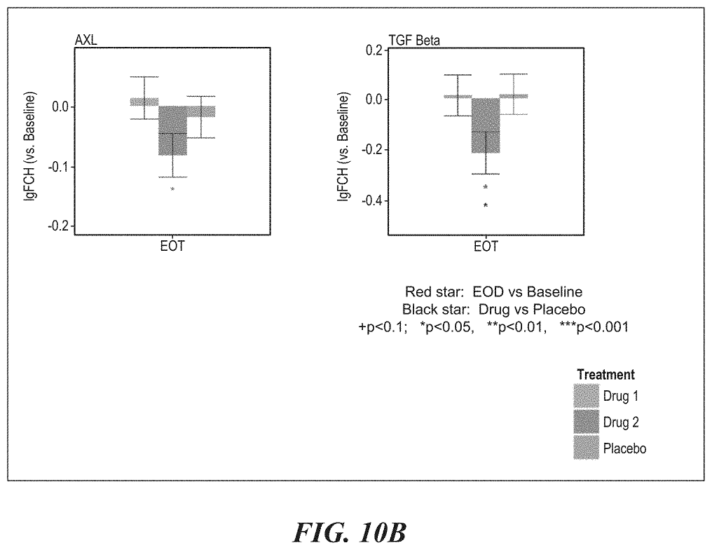

Bar graphs showing the relative fold change of selected biomarkers compared to baseline are shown in FIGS. 3-10. FIG. 3 depicts changes in general inflammation markers, FIG. 4 depicts changes in innate immunity related markers, FIGS. 5A and 5B depicts changes in T Cell/NK cell activation markers, FIGS. 6A and 6B depicts changes in Th1-related markers, FIGS. 7A and 7B depicts biomarker changes in Th17/Th1-related markers, FIG. 8 depicts marker changes Th2-related matters, FIG. 9 depicts maker changes in Th2 and other chemokines and adhesion molecules, and FIGS. 10A and 10B depicts changes in fibrotic and proliferative markers.

Patients in Treatment Group B: 2 g DS107 (i.e., Drug2g) exhibited clear differentiation from other two arms (i.e., Treatment Group A: 1 g DS107 and Treatment Group C: placebo) in serum inflammatory markers in terms of the number of differentially expressed genes and their significance. These differences were either signification or approaching significance for a number of inflammatory markers as well as fibrotic markers. Mechanistic analyses, including serum, are more sensitive in the detection of differences between arms, including in clinical scenarios where patient and site selection is not always ideal.

1-19. (canceled)

20. A method of treating a connective tissue disease or disorder in a subject in need thereof, the method comprising administering to the subject a composition comprising DGLA or a derivative thereof, 15-HETrE or a derivative thereof, or a combination thereof.

21. The method of claim 20, further comprising reducing an amount of transforming growth factor β (TGF-β) in the subject in need thereof.

22-25. (canceled)

26. A method of treating cancer in a subject in need thereof, the method comprising administering to the subject a composition comprising DGLA or a derivative thereof, 15-HETrE or a derivative thereof, or a combination thereof.

27. The method of claim 26, wherein the cancer is selected from the group consisting of renal cell carcinoma, hepatocellular carcinoma, cholangiocarcinoma, breast cancer, and cutaneous squamous cell carcinoma.

28. The method of claim 26, further comprising reducing an amount of TGF-β and/or estimated glomerular filtration rate (EGFR) in the subject in need thereof.

29. A method of treating a disease, a disorder, or a condition associated with T cell activation and/or mediated by CD40 in a subject in need thereof, the method comprising administering to the subject a composition comprising DGLA or derivative thereof, 15-HETrE or derivative thereof, or a combination thereof.

30. The method of claim 29, wherein the disease, disorder, or condition associated with T cell activation and/or mediated by CD40 is selected from the group consisting of multiple sclerosis, inflammatory bowel disease, hematologic malignancy, cancer, and immunosuppression.

31. (canceled)

32. The method of claim 30, wherein the hematologic malignancy is selected from the group consisting of Hodgkin's lymphoma, non-Hodgkin's lymphoma, B-cell lymphomas, lymphocytic leukemia, multiple myeloma, and acute myeloid leukemia.

33. (canceled)

34. The method of claim 29, further comprising reducing an amount of one or more cytokines and/or chemokines in the subject in need thereof.

35. The method of claim 34, wherein the one or more cytokines and/or chemokines are selected from the group consisting of interleukin 15 receptor α (IL-15RA), interleukin 2 (IL-2), interleukin 2 receptor β (IL-2Rβ), interleukin 7 (IL-7), chemokine ligand 25 (CCL-25), cluster of differentiation 6 (CD6), signaling lymphocytic activation molecule family member 1 (SLAMF1), cluster of differentiation 224 (CD244), and activated leukocyte cell adhesion molecule (ALCAM).

36. The method of claim 35, wherein T cell activation further comprises activation of one or more subtypes of T cells selected from the group consisting of Th1 T cells, Th17 T cells, and Th2 T cells.

37. The method of claim 36, wherein the T cell subtype is Th1 T cells and the one or more cytokines and/or chemokines are selected from the group consisting of chemokine ligand 16 (CCL-16), chemokine ligand 3 (CCL-3), chemokine C-X-C motif ligand 9 (CXCL-9), interleukin 18 (IL-18), interleukin 18 receptor 1 (IL-18R1), interleukin 18 binding protein (IL-18BP), chemokine ligand 4 (CCL-4), and chemokine ligand 3 (CCL-3).

38. The method of claim 36, wherein the T cell subtype is Th17 T cells and the one or more cytokines and/or chemokines are selected from the group consisting of peptidase inhibitor 3 (PI3), CUB domain-containing protein 1 (CDCP1), interleukin 17α (IL-17α), interleukin 17 D (IL-17D), interleukin 17β (IL-17β), interleukin 12β (IL-12β), interleukin 17 receptor α (IL-17Rα), interleukin 20 (IL-20), chemokine C-X-C motif ligand 1 (CXCL1), and interleukin 12 (IL-12).

39. The method of claim 36, wherein the T cell subtype is Th2 T cells and the one or more cytokines and/or chemokines are selected from the group consisting of interleukin 5 receptor α (IL-5Rα), interleukin 10 (IL-10), interleukin 10 receptor β (IL-10R13), and interleukin 13 (IL-13).

40. The method of claim 36, wherein the T cell subtype is Th2 T cells and the one or more cytokines and/or chemokines are further selected from the group consisting of chemokine ligand 11 (CCL-11), chemokine ligand 23 (CCL-23), chemokine ligand 24 (CCL-24), chemokine ligand 28 (CCL-28), interleukin 24 (IL-24), and VEGF-A.

41-45. (canceled)

46. A method of treating a fibrosis disease or disorder in a subject in need thereof, the method comprising administering to the subject a composition comprising DGLA or a derivative thereof, 15-HETrE or a derivative thereof, or a combination thereof.

47. The method of claim 46, wherein the fibrosis disease or disorder is selected from the group consisting of systemic fibrosis, renal fibrosis, lung fibrosis, skin fibrosis, myelofibrosis, and cardiac fibrosis.

48. (canceled)

49. The method of claim 47, wherein renal fibrosis further comprises glomerular diseases, tubulointerstitial disease, iatrogenic nephropathy, and/or renal ischemia.

50. The method of claim 49, wherein glomerular diseases are selected from the group consisting of focal segmental glomerulosclerosis, IgA nephropathy, crescentic glomerulonephritis, lupus nephritis, and diabetic nephropathy.

51. The method of claim 47, wherein lung fibrosis further comprises interstitial lung disease.

52. The method of claim 51, wherein lung fibrosis further comprises idiopathic pulmonary fibrosis, scleroderma, radiation fibrosis, iatrogenic, sarcoidosis, mixed connective tissue disease, polymyositis, dermatomyositis, and/or systemic lupus erythematosus.

53. The method of claim 47, wherein skin fibrosis further comprises scleroderma, systemic sclerosis, nephrogenic fibrosing dermopathy, mixed connective tissue disease, scleromyxedema, scleredema, eosinophilic fasciitis, and/or iatrogenic fibrosis.

54. The method of claim 46, further comprising reducing an amount of one or more cytokines and/or chemokines in the subject in need thereof.

55. The method of claim 54, wherein the one or more cytokines and/or chemokines are selected from the group consisting of TGF-β, PDGF, EGFR, VEGF-A, and AXL.

56. The method of claim 54, wherein the one or more cytokines and/or chemokines are TGF-β and PDGF.

57. The method of claim 54, wherein the one or more cytokines and/or chemokines are TGF-β, PDGF, VEGF-A, and EGFR.

58. The method of claim 54, wherein the one or more cytokines and/or chemokines are TGF-β, PDGF, and VEGF-A.

59. The method of claim 54, wherein the one or more cytokines and/or chemokines are TGF-β, PDGF, and EGFR.

60. The method of claim 54, wherein the one or more cytokines and/or chemokines is TGF-β.

61. The method of claim 20, wherein the composition is administered to the subject in an amount sufficient to provide up to about 8 g of DGLA or a derivative thereof per day.

62. The method of claim 20, wherein the composition is administered to the subject in an amount sufficient to provide between about 4 g and about 8 g of DGLA or a derivative thereof per day.

63. The method of claim 20, wherein the composition is administered to the subject in an amount sufficient to provide up to about 8 g of 15-HETrE or a derivative thereof per day.

64. The method of claim 20, wherein the composition is administered to the subject in an amount sufficient to provide between about 4 g and about 8 g of 15-HETrE or a derivative thereof per day.

65. The method of claim 20, wherein the composition is administered to the subject in an amount sufficient to provide up to about 8 g of DGLA or a derivative thereof per day and up to about 8 g of 15-HETrE or a derivative thereof per day.

66. The method of claim 20, wherein the composition is administered to the subject in an amount sufficient to provide between about 4 g and about 8 g of DGLA or a derivative thereof per day and between about 4 g and about 8 g of 15-HETrE or a derivative thereof per day.

67. The method of claim 20, wherein the composition is administered in 1 to 8 capsules per day.

68. The method of claim 20, wherein the composition is administered in 1 to 4 capsules per day.

69. The method of claim 20, wherein the composition is administered in 4 to 8 capsules per day.

70. The method of claim 67, wherein each capsule comprises up to about 1 g of DGLA or a derivative thereof and/or 15-HETrE or a derivative thereof.

71. The method of claim 20, wherein the composition is administered to the subject for a period of at least about 1 week, at least about 2 weeks, at least about 4 weeks, at least about 6 weeks, at least about 8 weeks, or at least about 10 weeks.

72. The method of claim 20, wherein the composition is not encapsulated.

73. The method of claim 20, wherein the composition is administered by oral administration.

74. The method of claim 20, wherein the composition is administered by topical administration.
